(19) United States
(12) Patent Application Publication
(10) Pub. No.: US 2026/0350794 A1
(43) Pub. Date: Nov. 5, 2026

(54) AN AUTOMATED METHOD FOR EVALUATING THEZONA PELLUCIDABINDINGCAPABILITY OF SPERMATOZOAIN CLINICAL ASSISTED REPRODUCTION

(71) Applicant: THE UNIVERSITY OF HONG KONG, Hong Kong (CN)

(72) Inventors: Philip Chi Ngong CHIU, Hong Kong (CN); William Shu Biu YEUNG, Hong Kong (CN); Erica Tsz Ying LEUNG, Hong Kong (CN); Lequan YU, Hong Kong (CN); Xianghan MEI, Hong Kong (CN); Ernest Hung Yu NG, Hong Kong (CN); Hang Wun Raymond LI, Hong Kong (CN)

(73) Assignee: THE UNIVERSITY OF HONG KONG, Hong Kong (CN)

(21) Appl. No.: 19/487,648

(22) PCT Filed: Jun. 24, 2024
(86) PCT No.: PCT/CN2024/101066

§ 371 Date: Nov. 25, 2025

Related U.S. Application Data
(60) Provisional application No. 63/511,375, filed on Jun. 30, 2023.
(60) Provisional application No. 63/567,147, filed on Mar. 19, 2024.

(51) Int. Cl. G06T 7/00; G06N 3/045; G06N 3/0464; G06N 3/088; G16H 10/40; G16H 30/40; G16H 50/20

(52) U.S. Cl. CPC G06T 7/0012; G06N 3/045; G06N 3/0464; G06N 3/088; G16H 10/40; G16H 30/40; G16H 50/20; G06T 2207/10056; G06T 2207/20081; G06T 2207/20084

(57) ABSTRACT

An automated method valuates the ZP-binding capability of spermatozoa according to their morphological features in assisted reproduction using deep learning. At least one sample of zona pellucida (ZP)-bound spermatozoa and at least one sample of ZP-unbound spermatozoa are obtained, and digital images of the ZP-bound and ZP-unbound spermatozoa are obtained. The digital images are processed according to at least one deep learning model configured to use the digital images to generate a binary prediction regarding a ZP-binding ability of spermatozoa, with the binary prediction being normal or defective ZP binding. A method also predicts fertilization success in clinical assisted reproduction based on the ZP-binding capability of spermatozoa.

### CROSS REFERENCE TO RELATED APPLICATIONS

This application is a U.S. National Stage Application under 35 U.S.C. § 371 of International Patent Application No. PCT/CN2024/101066 filed Jun. 24, 2024, which claims the benefit of priority of U.S. Provisional Application No. 63/511,375, filed Jun. 30, 2023, and U.S. Provisional Application No. 63/567,147, filed on 19 Mar. 2024, the disclosures of which are incorporated herein by reference in their entireties. The International Application was published on Jan. 2, 2025, as International Publication No. WO 2025/002066A1.

### 1. FIELD

Disclosed is an automated method for evaluating the zona pellucida (ZP)-binding capability of spermatozoa according to their morphological features in assisted reproduction using deep learning. Also provided is a method of predicting fertilization success in clinical assisted reproduction based on the ZP-binding of spermatozoa.

### 2. BACKGROUND

Human fertilization begins when a capacitated spermatozoon binds to the zona pellucida (ZP) of an oocyte. In fertile men, only a limited proportion of motile spermatozoa in the ejaculate (<7%) can bind to and penetrate the ZP. This observation can be attributed to the selective nature of ZP interacting with the sperm subpopulations characterized by normal chromatin structure, superior morphology, and fertilization potential.

Infertility is a global health concern affecting ~15% of heterosexual couples of reproductive ages, with approximately 30% of the cases categorized as male-factor infertility. Assisted reproductive treatments (ARTs) including conventional insemination in vitro fertilization (IVF) and intracytoplasmic sperm injection (ICSI) are commonly offered to patients with long-standing duration of infertility. However, 10% of all of the couples and 25% of all of the couples with unexplained infertility encountered fertilization failure following conventional insemination during IVF. ICSI involves direct injection of a morphologically normal and motile spermatozoon to bypass the binding between gametes and the subsequent penetration processes. This technique has greatly improved the fertilization rates for couples diagnosed with severe male factor infertility when compared with conventional insemination during IVF.

Conventional semen analysis according to the World Health Organization (WHO) Laboratory Manual for the Examination and Process of Human Semen is routinely used to establish the fertility profile of semen samples. The semen analysis result plays a crucial role in deciding between conventional insemination and ICSI. During the first IVF cycle, patients often undergo conventional insemination as the fertilization method unless semen analysis result indicates severe abnormalities. However, semen parameters such as motility and morphology do not necessarily reflect the fertilization potential of spermatozoa, and thus suboptimal fertilization outcome following conventional insemination may occur in men with semen parameters within the normal ranges. And the need for ICSI in the following cycles may inflict additional financial burdens on them.

Defective sperm zona pellucida (ZP) binding (DSZPB) is a major cause of low fertilization rates in conventional insemination during IVF. DSZPB is frequently found in patients with mild to moderate teratozoospermia, severe teratozoospermia and oligozoospermia. In addition, DSZPB also occurs in approximately 13% of men with normal semen parameters, who often go undetected during initial semen assessment due to the lack of direct correlation between the semen parameters and ZP-binding ability of spermatozoa. It is therefore imperative to develop a highly robust, automated method to evaluate the fertilization potential of spermatozoa before commencing IVF to improve the overall clinical management.

Deep learning is an advanced machine learning approach involving artificial neural networks (ANNs). Convolutional neural network (CNN) is a specialized class of deep learning that has been designed to analyze the structural elements of the datasets for image classification and object recognition. CNN is a highly complex and intertwined multi-layered network consisting of interconnected nodes within layers, which resembles the dynamic decision-making processes occurring within the neural networks of human brain. During training, CNN assigns weights to each identifiable feature according to their importance in relation to the final output. CNN can systematically analyze the input data through successive layers of data representations leading to a net result. Starting from the first layer, the model focuses on the low-level features such as the edges and corners to hierarchically collect the visual information from the datasets. As the learning period progresses, the model increases the complexity of the identification process by extracting high-level, discriminative features in subsequent layers. A well-trained model can generalize to unseen data collected from the same condition that was used to generate the training datasets.

Conventional semen analysis fails to provide comprehensive information regarding the fertilization potential of the spermatozoa in the samples. Spermatozoa from men with normal semen parameters can manifest functional defects and genetic abnormalities leading to suboptimal fertilization outcome. And the semen parameters vary greatly among patients, making it difficult to establish threshold values to predict fertility status with absolute certainty. Men with semen parameters below the reference values are not guaranteed to be infertile and vice versa. Thus, the clinical significance of the reference values is controversial. Furthermore, the manual assessment of semen parameters mostly relies on the clinical experience and judgement of the embryologist, which could result in high intra-and inter-laboratory variations. There is an urgent need for a highly robust, reliable, and automated method to evaluate the fertilization competence of semen samples according to their ZP-binding ability.

A known automated evaluation of sperm morphology is described in U.S. Patent Publication No. US20210374952A1, now U.S. Pat. No. 11,926,809B2, issued on Mar. 12, 2024, which is incorporated herein by reference in its entirety.

### 3. SUMMARY

The present invention provides a method to evaluate the ZP-binding ability of spermatozoa by diff-quik stained image analysis of sperm morphology using deep learning. ZP-binding ability is a quantitative, independent sperm quality metric related to fertilization and pregnancy outcomes following conventional insemination.

The present intervention allows real-time evaluation of sperm fertilization potential, independent of those derived from the standard semen analysis. The predicted percentage of ZP-binding ability generated by the model can identify normospermic patients (as determined by the conventional semen analysis) whose spermatozoa with defective ZP-binding ability. Such patients are at a higher risk of encountering fertilization failure following conventional insemination during IVF as a result of unsuccessful binding between gametes. The additional information provided by the present invention can potentially allow the patients to make informed decision on the fertilization method (conventional insemination vs. ICSI), preventing them from psychological and financial sufferings caused by fertilization failure following conventional insemination.

The present intervention is a novel method specifically designed to identify spermatozoa with/without ZP-binding ability according to their morphologic features using an advanced deep learning model. During the development stage, a representative database containing diff-quik stained images of ZP-bound spermatozoa and spermatozoa with defective ZP-binding ability has also been established, which could be accessible to the public for clinical and research uses.

Provided herein is the use of deep learning for morphologic prediction of ZP-bound and unbound spermatozoa by images analysis according to their morphologic features.

Also provided is a novel database containing diff-quik stained images of 1) laboratory samples of ZP-bound spermatozoa collected using a modified spermatozoa-ZP coincubation assay; and 2) clinical samples of spermatozoa with defective ZP-binding ability and zero fertilization rates, which are considered as the truly representative class of unbound spermatozoa. Considering the differences in the microenvironment between the laboratory and clinical samples, Cycle Generative Adversarial Network (CycleGAN) was used to translate the specific characteristics of the laboratory samples into the clinical samples to generate a more consistent dataset representative of sperm morphology. A novel, deep learning-based method was built using CNN named VGG-13 to identify the shared and unique morphological features within each class (ZP-bound and unbound spermatozoa) for plausible predictions of ZP-binding ability.

Provided herein is a deep learning-based method for assisted reproduction comprising:
a. obtaining at least one sample of zona pellucida (ZP)-bound spermatozoa and at least one sample of ZP-unbound spermatozoa;
b. obtaining one or more digital images of said (ZP)-bound and ZP-unbound spermatozoa through one or more imaging modalities; and
c. processing the one or more digital images according to at least one deep learning model configured to use sperm images to generate binary prediction regarding ZP-binding ability of spermatozoa, wherein the binary prediction is normal or defective ZP binding.

Also provided is a method of predicting fertilization success following conventional insemination during IVF, comprising:
a. obtaining a sperm sample from a subject undergoing IVF;
b. obtaining one or more digital images of said sperm sample through one or more imaging modalities; and
c. processing the one or more digital images according to at least one deep learning model to obtain a binary prediction regarding ZP-binding ability of said sperm sample, wherein normal ZP-binding of said sperm sample predicts fertilization success, and defective ZP-binding of said sperm sample predicts fertilization failure.

Also provided is a method for predicting the ZP-binding ability of spermatozoa, comprising:
a. obtaining at least one sample of ZP-bound spermatozoa and at least one sample of ZP-unbound spermatozoa;
b. obtaining one or more digital images of said ZP-bound and ZP-unbound spermatozoa through one or more imaging modalities; and
c. processing the one or more digital images according to at least one deep learning model configured to use sperm images to generate a binary prediction regarding ZP-binding ability of spermatozoa, wherein the binary prediction is normal or defective ZP binding, wherein said deep learning model is VGG-13 which predicts ZP-binding ability with at least about 90 % accuracy, specificity, sensitivity, or a combination thereof.

### 4. BRIEF DESCRIPTION OF FIGURES

FIG. 1 shows a schematic diagram of the spermatozoa-ZP continuous coincubation assay to collect ZP-unbound spermatozoa. After sperm purification, a group of 4 oocytes in 30 μL of 1 M sperm in a droplet was incubated with 1M/mL of motile capacitated spermatozoa for a 6-hr incubation at 37 °C in 5% CO₂. ZP-bound spermatozoa were then removed from the surface of the oocytes through vigorous aspiration or pipetting at 2, 4, and 6 hr. After every 2 hr., the same batch of oocytes are returned back to the incubation droplet. All the spermatozoa remaining in the incubation droplet after 6 hours were considered as the unbound spermatozoa. The collected ZP-unbound spermatozoa remaining in the droplet were allowed to air-dry and subjected to diff-quik staining. The images were captured at a magnification of 1000× under oil immersion.

FIG. 2 shows a schematic diagram of the spermatozoa-ZP coincubation assay to collect ZP-bound, acrosome intact spermatozoa. A group of 4 oocytes in 30 μL of 2M sperm in a droplet was incubated with 2M/mL of motile capacitated spermatozoa for 30 min to collect acrosome intact, ZP-bound spermatozoa. Removal of ZP-bound sperm was performed through vigorous pipetting. The collected ZP-bound spermatozoa were allowed to air-dry and subjected to diff-quik staining for further evaluation. The images were captured at a magnification of 1000× under oil immersion.

FIG. 3 shows the DNA integrity evaluation of spermatozoa by Terminal deoxynucleotidyl transferase mediated dUTP Nick End Labeling (TUNEL) and COMETassays. ZP-bound spermatozoa had a significantly lower percentage of DNA fragmentation rates. (n=5, *p<0.05).

FIG. 4 shows the chromatin integrity evaluation of spermatozoa by acridine orange staining. ZP-bound spermatozoa had a significantly higher percentage of chromatin integrity. (n=5, *p<0.05).

FIG. 5 shows the protamination evaluation of spermatozoa by chromomycin A3 (CMA3) staining. ZP-bound spermatozoa had a significantly higher percentage of protamination degree. (n=5, *p<0.05).

FIG. 6 shows the methylation evaluation of spermatozoa by immunofluorescent staining of 5-Methylcytosine (5-Mc). ZP-bound spermatozoa had a significantly higher percentage of methylation level. (n=5, *p<0.05).

FIG. 7 shows the morphology evaluation of spermatozoa by manual counting according to the WHO criteria. ZP-bound spermatozoa were found with a significantly higher percentage of normal morphology. (n=5, *p<0.05).

FIG. 8 shows the image processing and extraction procedures of individual sperm heads from the diff-quik stained images. Individual sperm heads were identified by the standard k-means clustering algorithm and cropped to the size of 128×128.

FIG. 9 shows the design of a hybrid AI model combining CNN and sparse neural network (SNN). Processed sperm images were the input data. Normal and defective ZP-binding ability were the output labels. The images were fed into ANNs consisting of successive hidden layers for image analysis. In the last layer, a final prediction score combining the scores from two ANNs was generated. The CNN receives a transformed image of spermatozoa, generated by a transformation and segmentation, at a plurality of layers including a first convolutional layer, a first pooling layer, a second convolutional layer, a second pooling layer, a first sparse and fully connected layer, and a second sparse and fully connected layer. The layers of the CNN process the transformed image to generate a prediction of Normal or Defective for the spermatozoa from the CNN. In parallel, a sparse neural network (SNN) receives a transformed image of spermatozoa generated by the transformation and segmentation, and the transformed image is processed using a feature extraction layer, an encoder dictionary which generates sparse codes, and a concatenate layer to generate a prediction of Normal or Defective spermatozoa from handcrafted features. The prediction from the CNN and the prediction from the SNN are combined to generate a final prediction of Normal or Defective for the spermatozoa.

FIG. 10 shows the learning performance of the hybrid AI model for binary classification of ZP-bound (recovered at 30 min) and -unbound spermatozoa. ZP-bound spermatozoa were found with significantly higher rates of DNA integrity, normal morphology, protamination and methylation when compared with the unbound spermatozoa. A) Cost curve: to evaluate the total error rates generated during training and validation B) Accuracy curve: to evaluate the classification accuracy of the model over the training period. A hybrid AI model shown in FIG. 9 achieved a validation accuracy of 81.6% and a validation cost of 0.480, indicating the morphological characteristics of the sperm subpopulations were distinctly identifiable by the model.

FIG. 11 shows the learning performance of the hybrid AI model for binary classification of clinical samples of spermatozoa with normal and defective ZP-binding ability. The hybrid model in FIG. 9 achieved a validation accuracy of 76.2% and a validation cost of 0.494, indicating the morphological characteristics of the sperm subpopulations were distinctly identifiable by the model.

FIG. 12 shows the development of a deep-learning based method to evaluate the ZP-binding ability of human spermatozoa. (A) The images were inputted into the VGG-13 model consisting of successive hidden layers for image analysis. Considering the limited availability of sperm images and the involvement of WHO criteria in SNN (human intervention), transfer learning using VGG-13 was chosen as the optimal technique to develop a deep-learning-based method for binary classification. To fine-tune the classifier while leveraging the capability of the pretrained VGG-13 model, the last layer of the network was modified while the previous layers were frozen with unchanged weights. (B) Accuracy curve: to evaluate the overall reliability of the model (Left). Cost curve: to measure the error rates of classifications (Right). (C) ROC curve: to examine the discriminative power of the model as reflected by the AUC value. (D) Confusion matrix: a performance measurement of the model for binary classification.

FIG. 13 shows the correlation between ZP-binding ability and fertilization rates following conventional IVF using the newly fine-tuned VGG-13 model. Diff-quik stained images of three groups of men with fertilization rates of 0%-40%, 41%-70% and 71%-100% were collected. The % ZP-binding ability per sample was determined using the VGG-13 model.

FIG. 14 shows the correlation between ZP-binding ability and fertilization rates following conventional IVF using the original VGG-13 model (without being fine-tuned with our database). Diff-quik stained images of three groups of men with fertilization rates of 0%-40%, 41%-70% and 71%-100% were collected. The % ZP-binding ability per sample was determined using the original VGG-13 model (without being fine-tuned with representative datasets).

FIG. 15 shows the process of determining the optimal cut-off value for clinical prediction using the newly fine-tuned VGG-13 model. ROC curve in logistic regression was established using the predicted % ZP-binding ability of high and low fertilization groups. At the selected threshold, the specificity was 93.0% and sensitivity was 91.7%.

FIG. 16 shows the comparison of clinical relevance between the conventional semen analysis and the present invention. Clinical data of ten patients undergoing conventional IVF were collected from an IVF clinic to further validate the clinical significance of the present invention. All patients recruited in this study had normal semen analysis results as determined by the embryologist and received conventional IVF as primary treatment. However, patients 1-4 failed to achieve high fertilization rates despite having normal semen analysis results (good motility and concentration), indicating the lack of correlation between these semen parameters and fertilization outcome. The residual sperm samples of these patients were further examined to evaluate the ZP-binding ability % using the present invention as described. The ZP-binding analysis results determined that patients 1-4 should not be recommended for IVF as the result of defective ZP-binding ability, correlating with the low IVF fertilization rates.

FIGS. 17A-17B show the image processing and extraction of individual sperm heads, with FIG. 17A showing identification of an individual sperm head by the standard k-means clustering algorithm and cropped to a pixel size of 128×128, using batch normalization and contrasting and identification of minimal area surrounding the sperm heads by the k-means clustering algorithm, and FIG. 17B showing representative images of a processed sperm head extracted from the raw images.

FIG. 18 shows the development of a VGG-13 model to evaluate the ZP-binding ability of human spermatozoa, with the images inputted into the VGG-13 model consisting of successive hidden layers for binary classification, and the VGG-13 model comprised a feature extractor and a classifier which were connected by an average pooling layer. The classifier includes a dropout layer and 3 fully connected layers. FIG. 18 also shows an accuracy curve to evaluate the overall reliability of the model, a cost curve to measure the error rates of classifications, a receiver operating characteristic (ROC) curve to examine the discriminative power of the VGG-13 model as reflected by the area under the ROC curve (AUC), and a confusion matrix to measure the classification performance of the model for binary classification.

FIG. 19 shows an evaluation of prediction reproducibility generated by the fine-tuned VGG-13 model, with four sets of processed samples collected and processed using established protocols described herein, with two individual slides of each sample inputted into the VGG-13 model to determine the predicted percentage of ZP-binding ability. The low variation in the predicted values between the slides indicates that the VGG-13 model is able to generate comparable outputs for different sets of images of the same image, indicating a high prediction reproducibility.

FIGS. 20A-20G show the saliency maps for representative images of human spermatozoa from the datasets of spermatozoa. The saliency map overlays highlighted the relevant features that contributed to the prediction output of the VGG-13 model, as shown by the pixel importance in orange. The overall percentage of pixel units localized to the anterior region relative to the entire sperm head was quantified using a predetermined image. FIGS. 20A-20B show spermatozoa with a high percentage of pixel importance clustering over the anterior regions. FIGS. 20C-20D show spermatozoa with an intermediate percentage of pixel importance clustering over the anterior regions. FIGS. 20E-20F show spermatozoa with a low percentage of pixel importance clustering over the anterior regions. FIG. 20G shows a comparison of the overall percentage of pixel importance of ZP-bound and unbounded spermatozoa from the entire dataset of spermatozoa.

FIG. 21 shows a positive correlation between ZP-binding ability and fertilization rates following conventional insemination in IVF using a newly fine-tuned VGG-13 model. Diff-quik stained images of three groups of men (n=165, p<0.001) with fertilization rates of 0%-40%, 41%-70%, and 71%-100% were collected as described herein. The percentage of ZP-binding ability per sample was determined using the fine-tuned VGG-13 model.

FIG. 22 shows the identification of an optimal cut-off value for clinical prediction using the newly fine-tuned VGG-13 model. The ROC curve in logistic regression was established using the predicted percentage of ZP-binding ability of high and low fertilization groups (n=113). At a selected threshold of 4.8%, the specificity was 93.8% and the sensitivity was 90.9% for the binary classification of ZP-binding ability.

FIG. 23 shows a comparison of clinical relevance between conventional semen analysis and the present invention, with clinical data of 30 patients undergoing conventional IVF being collected from an IVF clinic to further validate the clinical significance of the VGG-13 model. All patients recruited in the study had normal semen analysis results as determined by an embryologist, and received conventional insemination as a primary fertilization method. The residual sperm samples of the patients were further examined to evaluate the percentage of a predicted ZP-binding ability using the VGG-13 model.

FIG. 24 shows the collection of ZP-bound spermatozoa using a modified spermatozoa-ZP coincubation assay, with a group of 4 oocytes incubated with 2M/mL of motile capacitated spermatozoa for 30 minutes to collect acrosome intact, ZP-bound spermatozoa. The collected ZP-bound spermatozoa were allowed to air-dry and were subjected to Diff-Quik staining as described herein. The images of the spermatozoa were captured at a magnification of 1000× under oil immersion.

FIG. 25 shows representative sperm images of poor quality, with the images of extracted sperm heads examined to identify and remove those images with undesirable features from the datasets to ensure comprehensive representation of sperm morphology.

FIG. 26 shows a comparison of the training accuracy generated by the model at different learning rates (LRs), with the training accuracy deteriorated with increasing learning rates of 0.1 and 0.01, respectively, indicating the lack of convergence to optimal conditions during training. The learning rate of 0.001 was selected as the optimal learning rate for the VGG-13 model.

FIGS. 27A-27B shows the development of a basic CNN-based method for binary classification as the VGG-13 model to evaluate the ZP-binding ability of human spermatozoa, with the images inputted into the CNN-based VGG-13 model shown in FIG. 27A consisting of two convolutional layers and two max pooling layers as successive hidden layers for binary classification, and the VGG-13 model comprised a feature extractor and a classifier which were connected by an average pooling layer. The classifier includes a dropout layer and 3 fully connected layers. FIG. 27B also shows an accuracy curve to evaluate the overall reliability of the model, a cost curve to measure the error rates of classifications, a receiver operating characteristic (ROC) curve to examine the discriminative power of the VGG-13 model as reflected by the area under the ROC curve (AUC), and a confusion matrix to measure the classification performance of the model for binary classification.

FIGS. 28A-28B show a comparison and evaluation of a generalization ability between a basic CNN and the fine-tuned VGG-13 model, with both models individually tested on two independent sets of test data of ZP-bound spermatozoa (n=122) and unbound spermatozoa (n=98) for binary prediction. As shown in FIG. 28B, the basic CNN model misclassified a large proportion of spermatozoa in the unbound sperm dataset as the ones with ZP-binding ability, indicating the lack of the CNN model of good generation ability to unseen data for prediction.

FIG. 29 shows a correlation between ZP-binding ability and fertilization rates following conventional IVF using the original VGG-13 model (without being fine-tuned with the datasets). Diff-Quik stained images of three groups of men (n=101) with fertilization rates of 0%-40%, 41%-70%, and 71%-100% were collected. The predicted percentage of ZP-binding ability per sample was determined using the original VGG-13 model (without being fine-tuned with the representative datasets).

FIG. 30 shows a schematic of a system according to an implementation.

FIG. 31 shows a schematic of a computing device used in the implementation of the system in FIG. 30.

FIG. 32 shows a schematic of modules used in the implementation of the system in FIG. 30.

FIG. 33 shows a schematic of a classifier according to an alternative implementation.

FIG. 34 shows a flowchart of a method of collecting spermatozoa and capturing images of the collected spermatozoa.

FIG. 35 shows a flowchart of a method of operation of the system in FIG. 30.

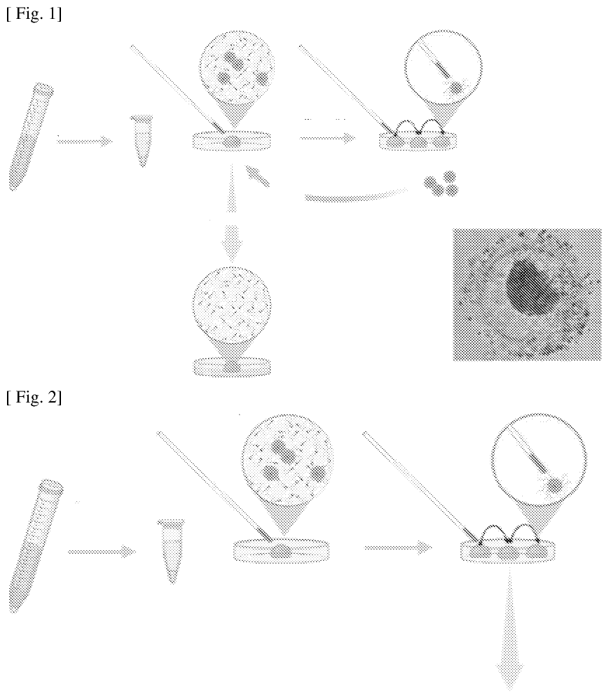

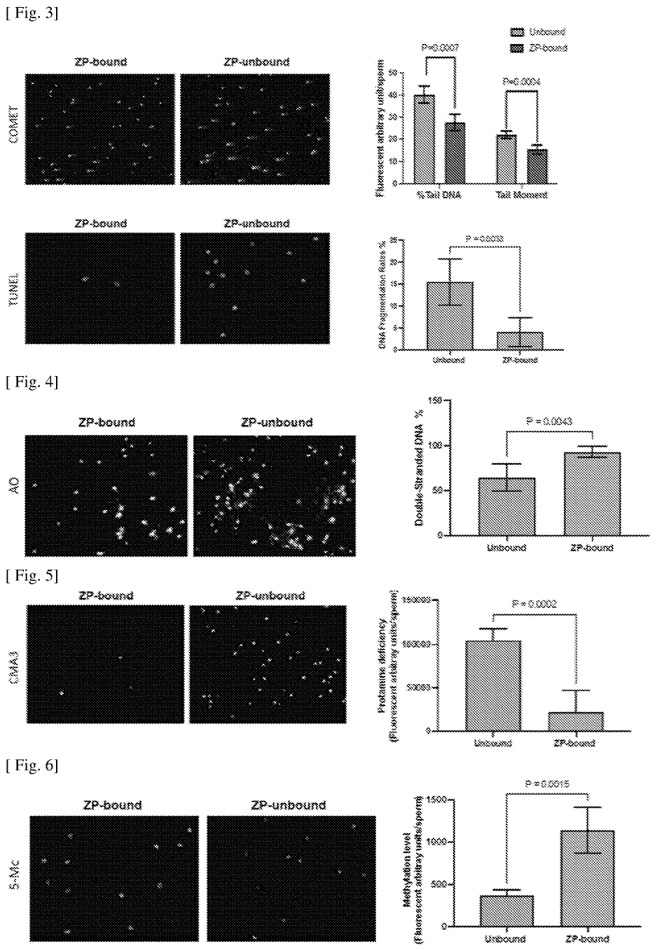

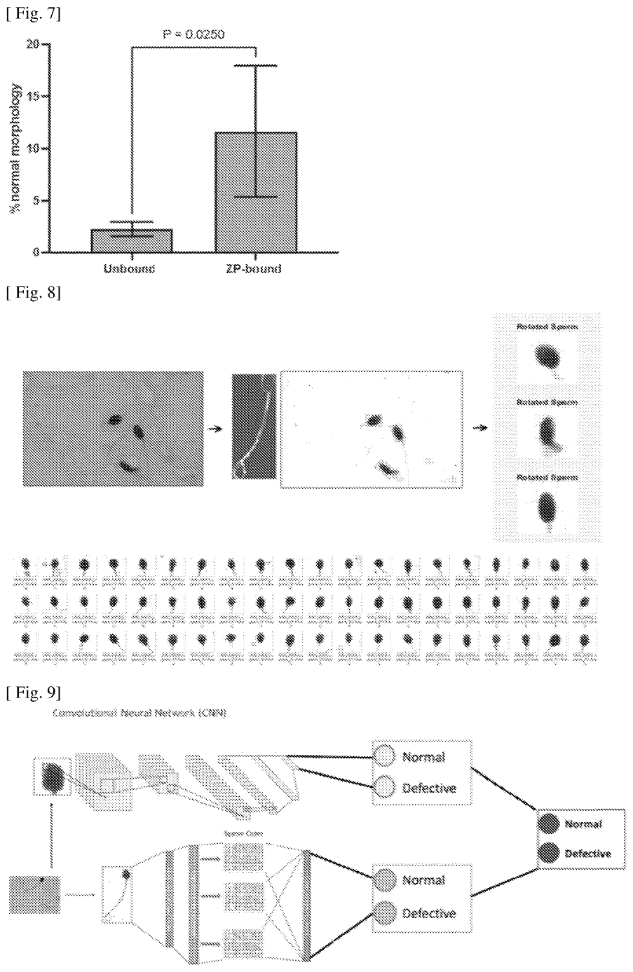

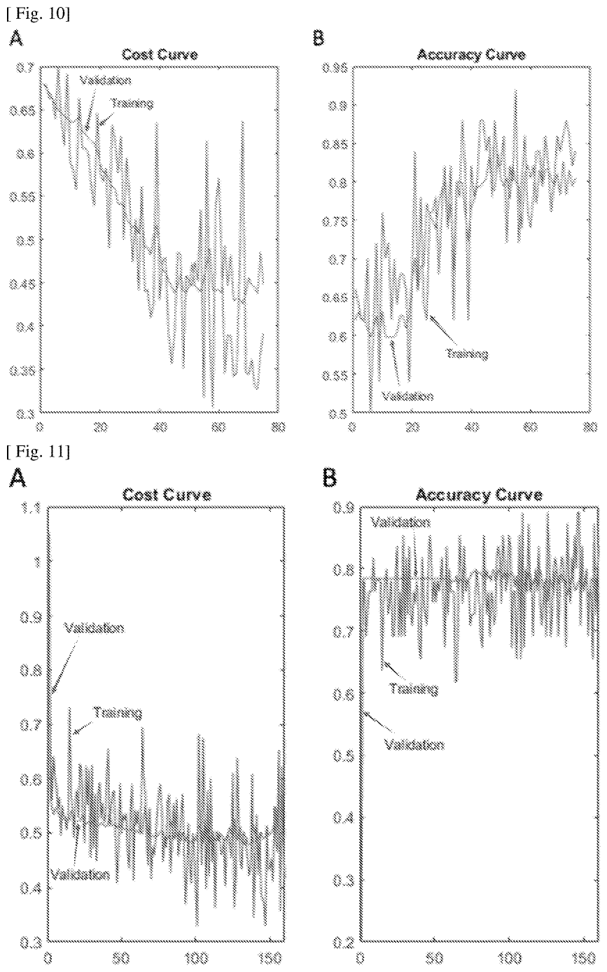

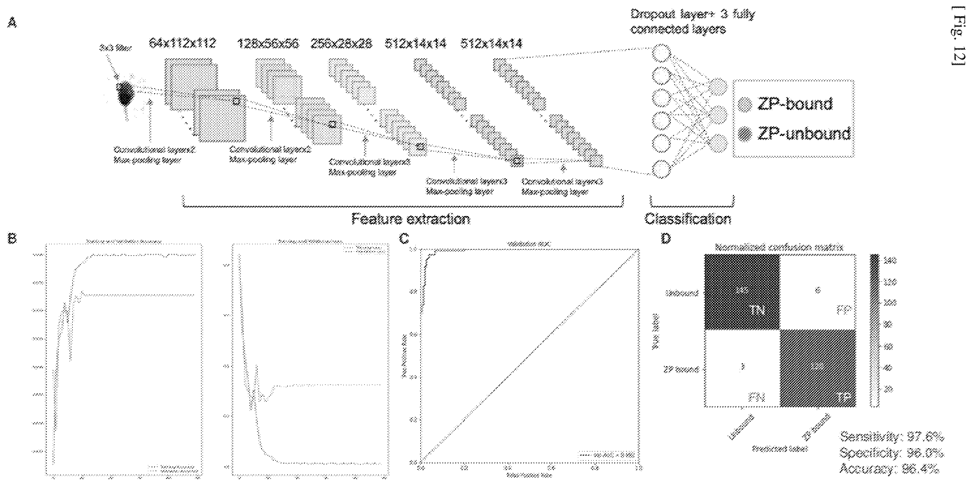

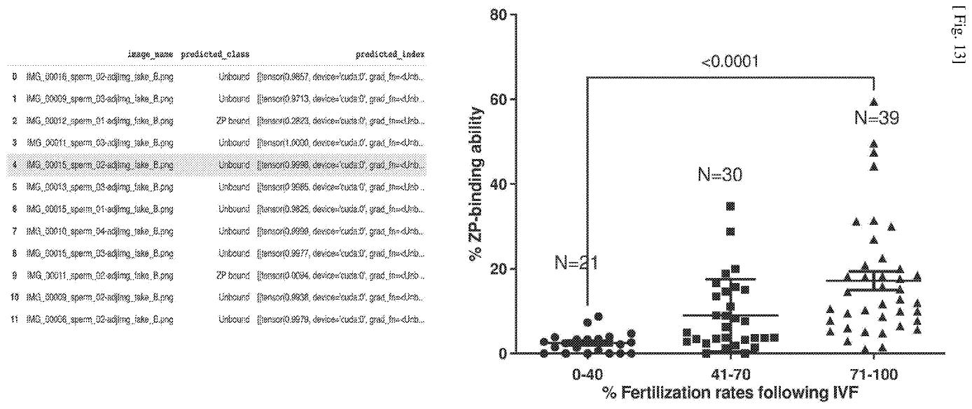

### 5. DETAILED DESCRIPTION

The model demonstrated success in classifying the sperm subpopulations with high specificity of 96.0% and sensitivity of 97.6%. Three groups of men with fertilization rates (FR) of 0%-40%, 41%-70% and 71%-100% were recruited from an assisted reproduction program to examine the correlation between the ZP-binding ability and fertilization rates of IVF. The predicative power of the model was tested on a total of 100 clinical samples from the aforementioned fertilization groups. High fertilization group (71%-100%) had a higher percentage of ZP-binding ability than the low fertilization group (0%-40%). The calculated threshold value to distinguish between normal and defective ZP binding ability of the sperm samples was 4.78% (where specificity: 93.0% and sensitivity: 90.5%), which can be used for clinical prediction of ZP-binding ability from morphological assessment at the single-cell level.

The present disclosure is to evaluate the percentage of ZP-binding ability of the diff-quik stained sperm samples according to their morphological features using deep learning and patients with percentage of ZP-binding ability below the threshold value of 4.78% will be recommended to undergo ICSI to improve the fertilization rates.

The following Examples further illustrate the findings of the present disclosure.

### 6. EXAMPLES

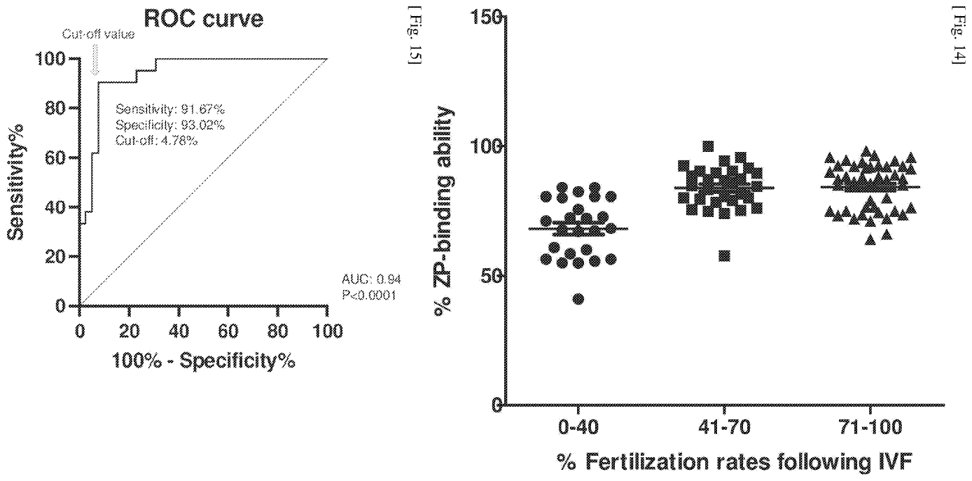

ZP-bound spermatozoa were collected by a previously modified spermatozoa-ZP coincubation assay, shown in FIG. 11. Clinical samples of spermatozoa with defective ZP-binding ability as reflected by complete failure of fertilization following conventional insemination during IVF collected from the IVF laboratory were considered the truly representative class of unbound spermatozoa. Diff-quik-stained images of ZP-bound and unbound spermatozoa were processed by K-means clustering algorithm for color-based segmentation to extract individual sperm heads from the background, as shown in FIG. 8. All the extracted sperm heads were cropped to a pixel size of 128×128 and converted to greyscale for training, as shown in FIG. 8.

ZP-bound and unbound spermatozoa were collected respectively under laboratory and clinical conditions, which could result in subtle microenvironmental differences that are undetectable by naked eye but can greatly confuse the deep learning classifier to generate misinterpretation of the datasets during training and validation. To remedy this issue, Cycle Generative Adversarial Network (CycleGAN) was used to translate the distinctive features of the laboratory samples into the clinical samples to generate a converted batch of datasets resembling the microenvironment of the laboratory samples. The present invention was built using an advanced deep learning architecture named VGG-13 in Python (3.10.6). VGG-13 was pretrained on ImageNet 1000 predefined classes and further finetuned with the newly established database containing a total 1,094 diff-quik-stained images of ZP-bound and unbound spermatozoa while 80% of the entire dataset was allocated to the training set. 20% of the entire dataset was allocated to the validation set. VGG-13 model was trained on datasets gather from different conditions (laboratory samples vs. clinical samples). All the clinical images were converted using cycle GAN model before training and testing. To finetune the classifier for the specific downstream task of sperm classification while leveraging the capability of the pretrained VGG-13 model, the last layer of the network was modified while the previous layers were frozen with unchanged weights. The numbers of channels increase by doubling the previous number every two layers (ranging from 64 to 512) with a max pooling layer between each increase. During training and validation, the input data (pre-processed, labelled images) was inputted into the VGG-13 network consisting of 10 convolutional layers which generated individual scores contributing to the final predictions (ZP-bound or unbound spermatozoa) over 50 epochs. Forward-propagation was performed to process the input data through successive hidden layers with 3×3 convolution filters by multiplying the weights to the node values to determine the node values in the next layer. More specifically, the interconnected nodes are activated when the output values of any individual node in the previous layer was above the threshold value. Additional data argumentations including horizontal flipping and random resizing were applied to further enhance the effective size and qualities of the training datasets. Back-propagation was simultaneously performed to adjust the weights on individual nodes to minimize the error rates. In the fully connected layers, the combined values generated by the model were the final predictions. The hyperparameters of the model were calibrated as follows: 1) maximum number of epochs: 50 epochs; 2) batch-size: 4; and 3) Learning Rate: 1××10⁻¹.

The newly fine-tuned VGG-13 model (constructed as shown in FIG. 12A) demonstrated success in classifying ZP-bound and unbound spermatozoa with a high accuracy and a low cost (a measure of the total error rates) during training and validation (FIG. 12B). The model also exhibited a high discriminative power to distinguish between the ZP-bound and unbound spermatozoa according to their morphological features, as reflected by the area under the operating characteristic (ROC) curve (AUC=0.992) (FIG. 12C). The classification performance of the model was evaluated in terms of accuracy (96.4%), specificity (96.0%), and sensitivity (97.6%) by the confusion matrix (FIG. 12D).

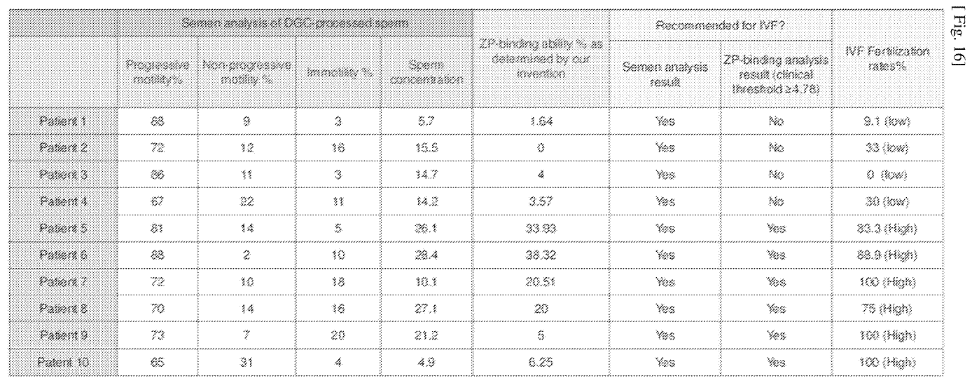

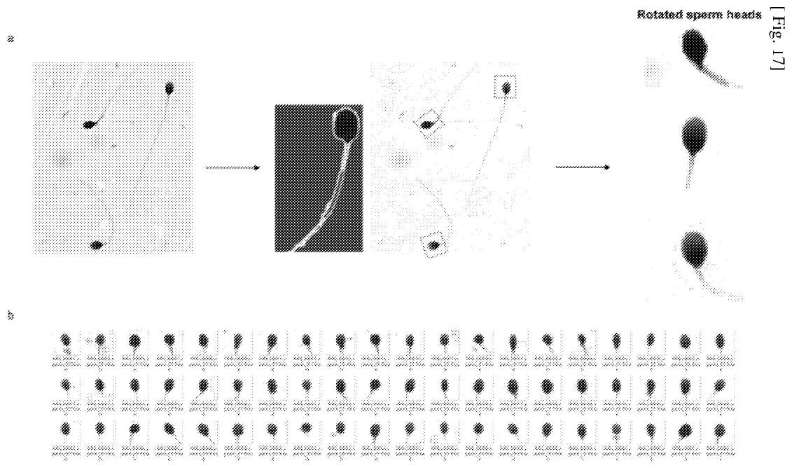

To examine the predictive power of the model for fertilization outcome, diff-quik-stained images of three fertilization groups with IVF fertilization rates of 0%-40%, 41%-70% and 71%-100% were inputted into the model for testing. The overall percentage of ZP-binding ability per sample was correlated with fertilization rates following IVF (FIG. 13). Further validation was conducted to compare the classification efficiency of ZP-binding ability between the present invention and original VGG-13 model (without being subjected to the fine-tuning process using the datasets). The results indicated that the original VGG-13 model lacked the capability to distinguish ZP-bound spermatozoa from the unbound ones, resulting in high percentage of ZP-binding ability in all fertilization groups (FIG. 14). The overall predictive power of the newly fine-tuned VGG-13 model was more superior to that of the original model. ROC in logistic regression was used to estimate the AUC (0.094) and P (<0.0001) values as a measure of discrimination between the high and low fertilization groups, as shown in FIG. 15. Youden's index was used to identify the optimal cut-off value (where specificity=sensitivity) on the ROC curve generated by the data. The cut-off value of 4.78% (where specificity: 93.0% and sensitivity:
90.5%) is the clinical threshold to distinguish between normal and defective ZP binding ability of the semen samples.

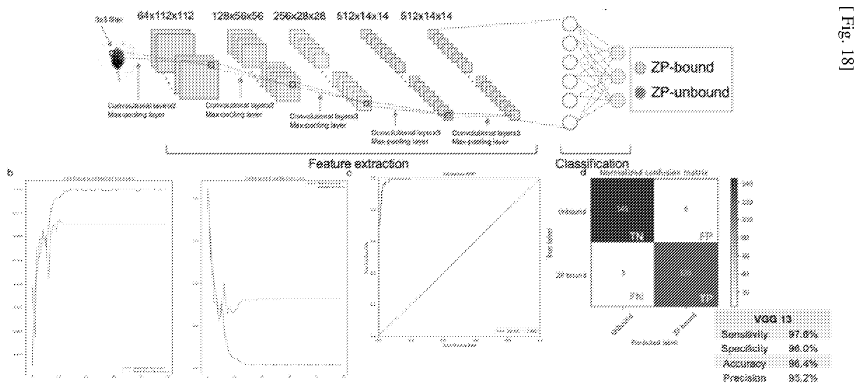

The clinical data of ten patients undergoing conventional IVF were selected to examine the clinical significance between the conventional semen analysis and the present invention, as shown in FIG. 16. All ten patients had normal semen analysis results as determined by the embryologist and received conventional IVF as primary treatment. Consistent with the normal semen analysis results, the percentage of ZP-binding ability of patients 5-10 with high fertilization rates (71-100%) following conventional IVF were above the clinical threshold (4.78%) as determined by the present invention. However, patients 1-4 failed to achieve high fertilization rates despite having normal semen analysis results. The present invention determined that these patients had a low percentage of ZP-binding ability (likely to fail IVF due to defective ZP-binding ability) and they should have had received ICSI instead of IVF as the fertilization method to improve the chances of fertilization success. The conventional semen analysis lacks the discriminatory power to identify patients with defective ZP-binding ability who require more invasive treatments instead of conventional IVF during initial screening. On the other hand, the preliminary results demonstrated the high prognostic value of the present invention when compared with the conventional semen analysis results alone. To put it another way, the present invention can serve as part of the semen analysis to provide supplementary information regarding the fertilization potential of semen samples, especially in cases where patients appear to have normal semen results but in fact with defective ZP-binding ability.

### 7. SUPPLEMENTAL IMPLEMENTATIONS

In routine semen analysis, sperm morphology is manually evaluated as one of the surrogate markers of male fertility potential. However, the predictive power of sperm morphology for fertilization and pregnancy outcome of in-vitro fertilization remain controversial mainly due to its high inter-and intra-assay variations. To address the limitations of the traditional method of assessing sperm morphology, a novel deep-learning model for identification of spermatozoa is provided with the ability of binding to the zona pellucida (ZP) of human oocytes, which is the first step in fertilization. A VGG-13 model was fine-tuned to classify a total of 1,083 images of ZP-bound and unbound spermatozoa according to their distinctive morphological features with a high accuracy (96.4%), specificity (96.0%), and sensitivity (97.6%). This model was validated on over 40,000 sperm images collected from three groups of infertile men (n=165) with fertilization rates ranging from 0-40% (low), 41-70% (intermediate) and 71-100% (high) following in-vitro fertilization with conventional insemination. Using images of spermatozoa from 113 infertile men with high and low fertilization rates in the aforementioned groups, a threshold of 4.8% for sperm morphology was established using receiver operating characteristic curve (Area under the curve: 0.970), which could differentiate samples with spermatozoa exhibiting normal from defective ZP binding ability with a high specificity of 93.8% and a sensitivity of 90.9%. In conclusion, the fine-tuned VGG-13 model is a highly robust and automated method for evaluation of sperm fertilizing ability.

Spermatozoa and oocyte collection is performed using collected residual semen samples from men. Normospermic samples were selected according to a predetermined WHO criteria, fifth edition 6: total volume≥1.5 mL, total motility≥40%, progressive motility≥32%, total sperm count≥39×106 per ejaculate, concentration≥15×106/mL, viability≥58%, and morphology≥4%. Direct swim-up method was used to isolate motile and viable spermatozoa from the seminal plasma as described previously 28. All processed spermatozoa were incubated in Earles balanced salt solution (EBSS) supplemented with 3% BSA (EBSS/3%BSA) to induce capacitation for subsequent coincubations 29. For clinical analysis, density gradient centrifugation (DGC) was used to process the clinical semen samples by centrifugation through sequential layering of two colloid media of different densities (40%/80%).

Immature oocytes (germinal vesicle/metaphase I oocytes) or mature metaphase II oocytes that failed to be fertilized following ICSI are obtained. Morphologically normal oocytes were stored in high-salt oocyte storage buffer containing 1.5 M MgC12, 0.1% polyvinyl pyrrolidone and 40 mM HEPES with pH 7.2 at 4 °C until use.

In performing a collection of data in a stored dataset, laboratory samples of ZP-bound human spermatozoa are obtained. Acrosome-intact, ZP-bound spermatozoa were collected by our modified spermatozoa-ZP coincubation assay as show in FIG. 24. In brief, 4 human oocytes were co-incubated in a 30 μL droplet of EBSS/3% BSA containing 2×106 spermatozoa covered with mineral oil at 37 °C in 5% CO2 for 30 min. After incubation, the oocytes were successively washed in 3 droplets of EBSS/no BSA to dislodge loosely bound spermatozoa. The ZP-bound spermatozoa were then removed from the surface of the oocytes by vigorous aspiration using a fine-bored glass pipette in a confined area containing 10 μL of EBSS without BSA on a sterile glass slide.

Clinical samples of ZP-unbound human spermatozoa and patients are allocated to three groups of fertilization rate. Normospermic samples with defective ZP-binding ability, as evidenced by complete fertilization failure following conventional insemination in IVF and the absence of ZP-bound spermatozoa in the inseminated oocytes, are considered to be the truly representative class of ZP-unbound spermatozoa. Three groups of samples with fertilization rates (FR) of 0-40%, 41-70% and 71-100% were collected to examine the correlation between ZP-binding ability and fertilization rates of conventional insemination in IVF, and to identify a clinical threshold distinguishing between normal and defective ZP binding ability of the processed sperm samples.

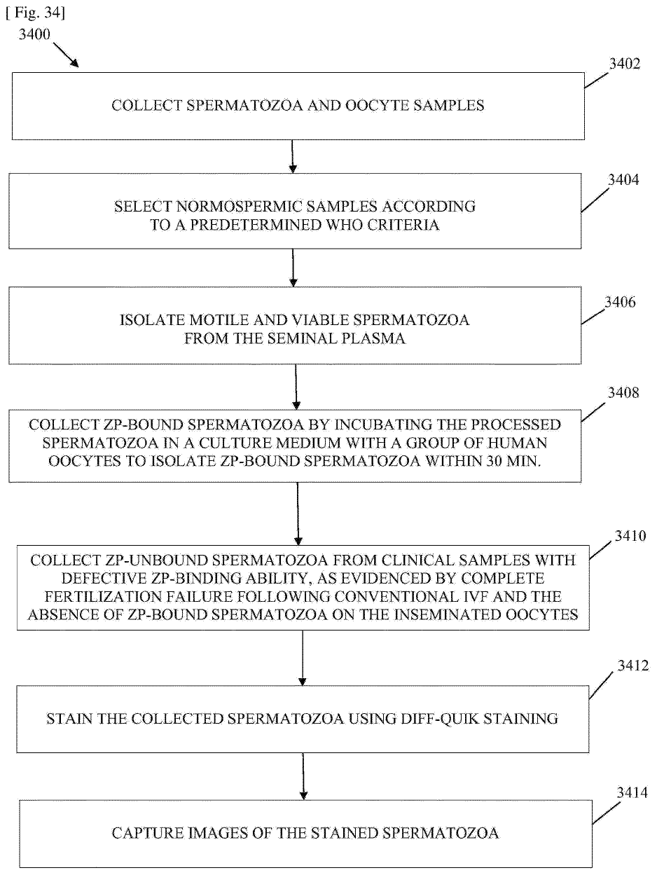

Diff-Quik staining of spermatozoa is performed such that all collected spermatozoa were subjected to Diff-Quik staining. In brief, air-dried spermatozoa were fixed with a methanol-based fixative solution for 15 sec. and stained with the staining solution 1 (buffered solution of Eosin Y) for 10 sec. followed by the staining solution 2 (buffered solution of thiazine dyes) for 10 sec. The excess solution was allowed to drip off the slides between steps. Diff-Quik stained images of spermatozoa were captured with oil immersion under a light microscope (Zeiss, Gottingen, Germany) at a magnification of 1000×. The collection of spermatozoa and oocyte samples and the capturing of images of the stained spermatozoa are described below with reference to FIG. 34.

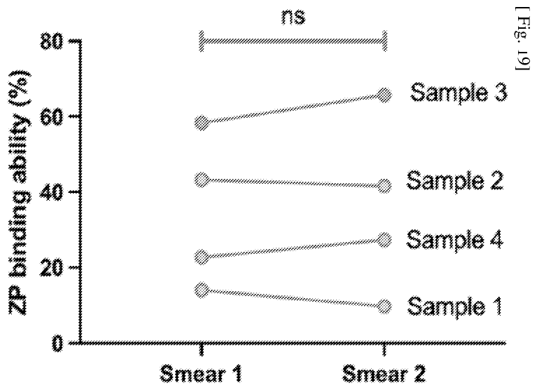

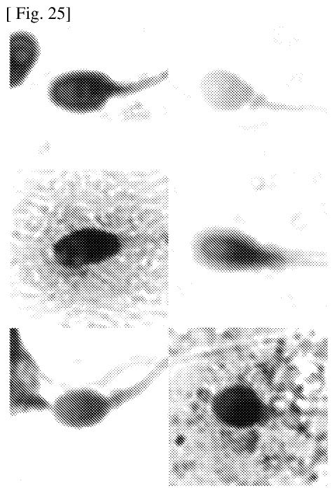

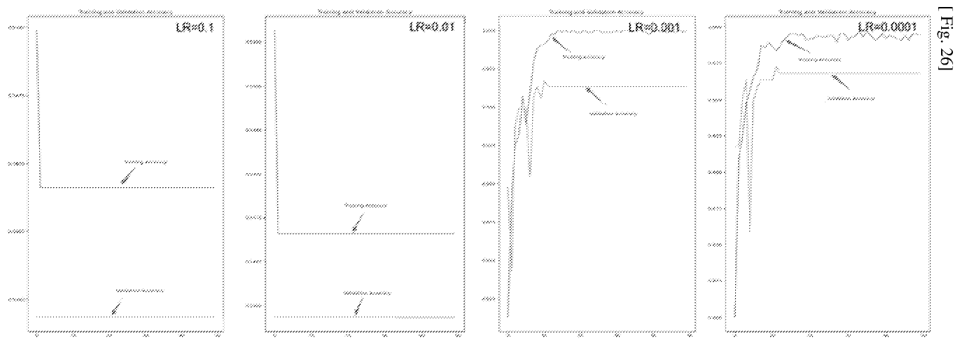

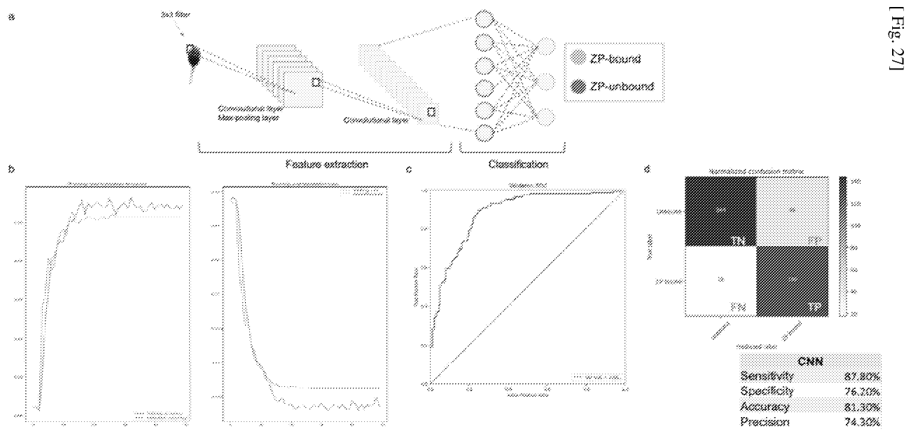

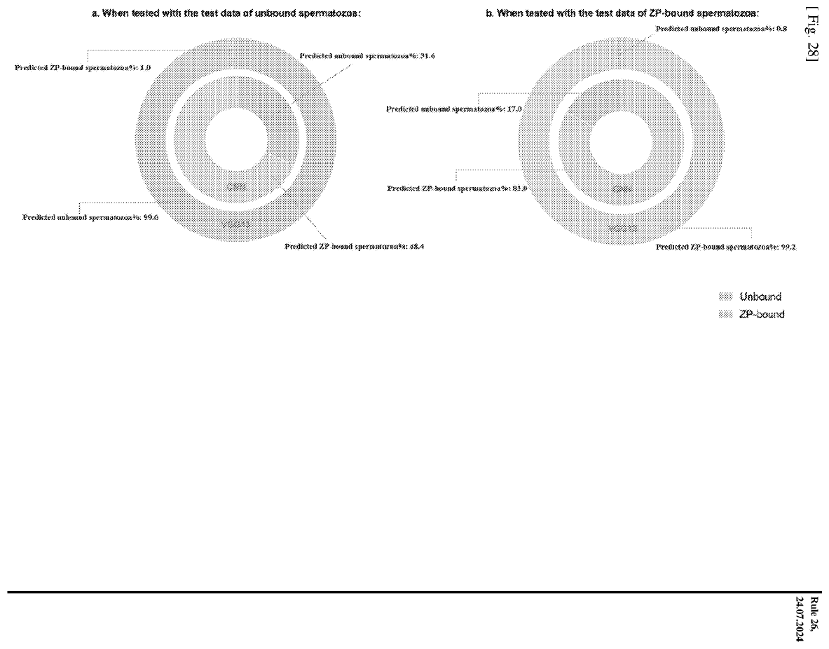

The disclosed invention then uses a disclosed deep learning-based method having an advanced CNN architecture named VGG-13 pretrained on ImageNet 1,000 predefined classes and further fine-tuned with a newly established database containing a total of 1,083 Diff-Quik-stained images of ZP-bound and unbound spermatozoa. All Diff-Quik stained images of spermatozoa were processed by a known K-means clustering algorithm for color-based segmentation to extract and identify individual sperm heads from the background, as shown in FIG. 17A. The extracted sperm heads were cropped to a pixel size of 128×128, turned to an up-right position and converted to grey scale before use, as shown in FIG. 17B illustrating representative images of processed sperm heads extracted from the raw images. To prevent the model from being misled and identifying irrelevant information during training, images of poor quality spermatozoa in the dataset including those with debris and/or a dirty background were manually removed from the dataset, as shown in FIG. 25. Clinical conditions might result in subtle microenvironmental differences that were undetectable by naked eye but could greatly confuse the deep learning classifier to misinterpret the datasets during training and validation. To remedy this issue, Cycle Generative Adversarial Network (CycleGAN) was used to translate the distinctive features of the laboratory samples into the clinical samples to generate a converted batch of datasets resembling the microenvironment of the laboratory samples. More specifically, all clinical samples used in this study were subjected to an additional step of image processing by CycleGAN for image conversion. 80% of the entire dataset was randomly allocated to the training set, and the remaining 20% of the entire dataset was allocated to the validation set. Additional 220 images were collected as the test set to examine the generalization ability of the model after fine-tuning.

The disclosed VGG-13 model was implemented in PyTorch. The deep learning algorithms were adapted from the transfer learning tutorial on PyTorch by Sasank Chilamkurthy. In VGG-13, the numbers of channels increased by doubling the previous number every two layers (ranging from 64 to 512) with a max pooling layer of 2×2 between each increase. During training and validation, the input data was fed into the VGG-13 network consisting of 10 convolutional layers which generated individual scores contributing to the final predictions over 50 epochs. The model consisted of two components, namely a feature extractor and a classifier, which were linked by an average pooling layer. To finetune the classifier for the specific downstream task of sperm classification while leveraging the capability of the pretrained VGG-13 model, the last layer of the network was modified while the previous layers were frozen with unchanged weights. In one implementation, the classifier is configured as shown in FIG. 33. The classifier was composed of three linear layers, where the first two were followed by a rectified linear unit (ReLU) layer and a dropout layer in a sequential manner. In the last fully connected linear layer, the number of output feature was modified from 1,000 to 2 for binary classification. Forward-propagation was performed to process the input data through successive hidden layers with 3×3 convolution filters by multiplying the weights to the node values to determine the node values in the next layer. More specifically, the interconnected nodes were activated when the output values of any individual node in the previous layer were above the threshold value. Additional data argumentations including horizontal flipping and random resizing were applied to further enhance the effective size and qualities of the training datasets. Stochastic Gradient Descent (SGD) was simultaneously performed to adjust the weights on individual nodes to minimize the error rates. Cross Entropy Loss was used to measure the difference between predicted probabilities and true class labels. The hyperparameters of the model were calibrated as follows: 1) maximum number of epochs: 50 epochs; 2) batch-size: 4; and 3) learning rate: 1×10⁻². The model was developed and deployed using the GOOGLE Colab with NVIDIA Tesla P100 or NVIDIA Tesla V100. Custom code was specific to the computing equipment employed for image processing and binary classification.

A confusion matrix was used to evaluate the model performance for binary classification in terms of accuracy, specificity, sensitivity, precision, and recall. The area under the receiver operating characteristic (ROC) curve (AUC) was used to evaluate the discriminative power of the model to distinguish the ZP-bound and the unbound spermatozoa according to their morphological features. Saliency mapping was used to identify the pixels that were most relevant to the classification and to determine the most significant region of the images. ROC in logistic regression was used to estimate the AUC and P values as a measure of discrimination between the high and low fertilization groups of clinical samples. The Youden's index (sensitivity+specificity-1) was used to identify the optimal threshold on the ROC curve generated by the data. Statistical software such as GraphPad Prism 9.1.0, GraphPad software, CA, USA, was used to analyze the clinical data. Two-tailed unpaired t-test was used to examine the differences between three groups of fertilization rate. If the data failed the normality test, Mann-Whitney (non-parametric) test was used to perform statistical analysis. A probability value of <0.05 was considered as statistically significant.

The VGG-13 model was fine-tuned using the database containing a total of 1,083 of Diff-Quik-stained images of 1) ZP-bound spermatozoa: laboratory samples of spermatozoa binding to the ZP of oocytes in the modified spermatozoa-ZP co-incubation assay; and 2) unbound spermatozoa: clinical samples of spermatozoa with defective ZP-binding ability resulting in fertilization failure after conventional insemination. Transfer-learning allowed the existing VGG-13 model to retain previously acquired information from the ImageNet 1,000 predefined classes for feature extraction and adapt itself to a new task by adjusting its parameters on the dataset of Diff-Quik-stained spermatozoa. The newly fine-tuned VGG-13 model, constructed as shown in FIG. 18A, demonstrated success in classifying the ZP-bound and the ZP-unbound spermatozoa with a high accuracy and a low cost (a measure of the total error rates) during training, as shown in FIG. 18B, without displaying any indications of overfitting over 50 epochs. The model was trained on the datasets using a batch size of 4 with a learning rate ranging from 0.0001 to 0.1, as shown in FIG. 26. The results indicated that a high learning rate negatively impacted the weight adjustment with respect to a loss gradient during training, and therefore, 0.001 was selected as the optimal learning rate for the model, as reflected by the high accuracy, as shown in FIG. 26.

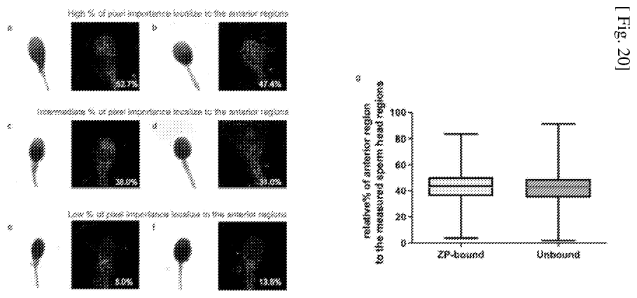

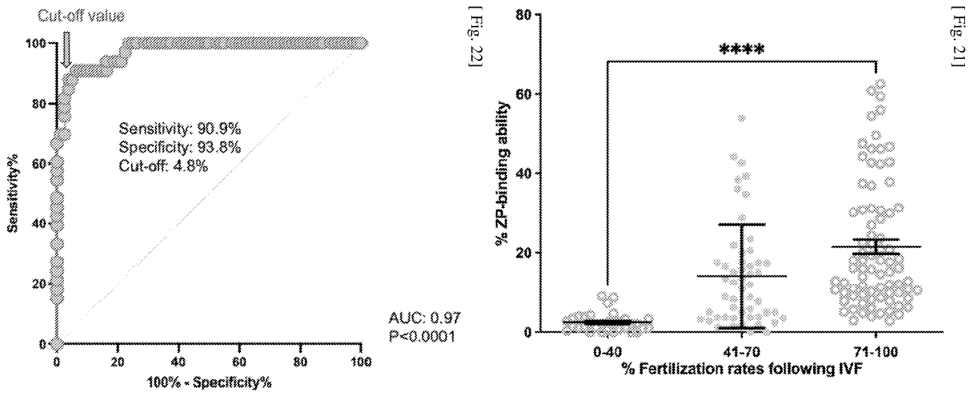

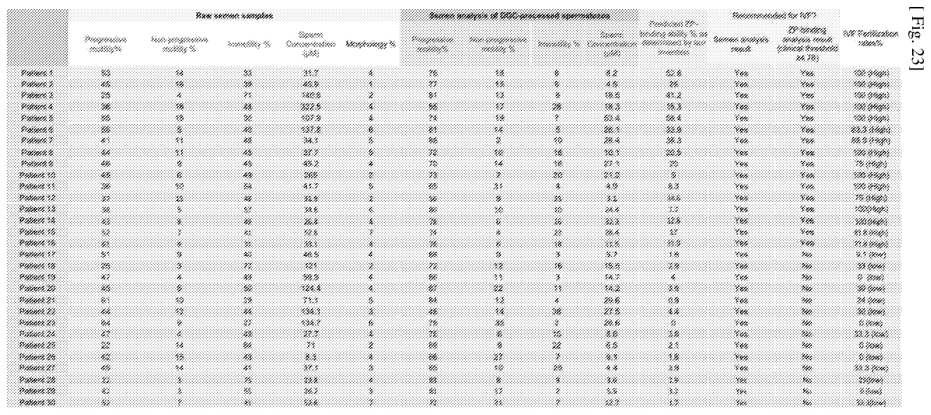

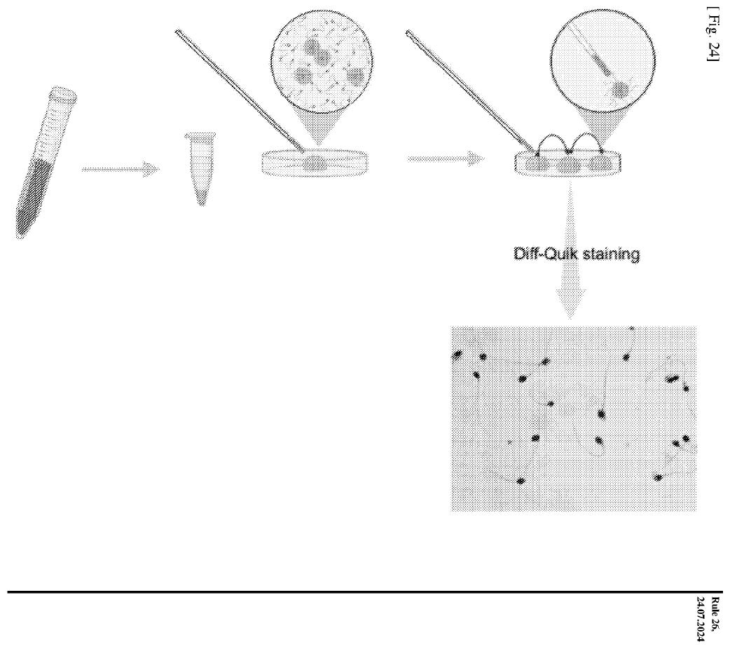

The model discriminated the ZP-bound spermatozoa from the unbound spermatozoa according to their morphological features with an AUC of ROC curve of 0.992, as shown in FIG. 18C. The model achieved good classification performance with high accuracy (96.4%), specificity (96.0%), and sensitivity (97.6%) as determined by the confusion matrix, as shown in FIG. 18D. The observed variations across two individual sets of images of samples were low, demonstrating that the model had a good prediction reproducibility, as shown in FIG. 19. Saliency maps such as shown in FIGS. 20A-20F indicated that the model consistently focused on regions corresponding to the sperm head and mid-pieces in all images, with minimal attentions to the background noise and debris. The analysis of the heat maps generated using the training datasets indicated that the model primarily emphasized on the anterior regions of the sperm heads, as shown in FIG. 20G, with 43.0%±5.0% in both the ZP-bound and the unbound spermatozoa. Overall, the saliency maps indicated that the model specifically detected and recognized the distinct features of sperm heads, shedding light on the decision-making process used within the black-box approach.

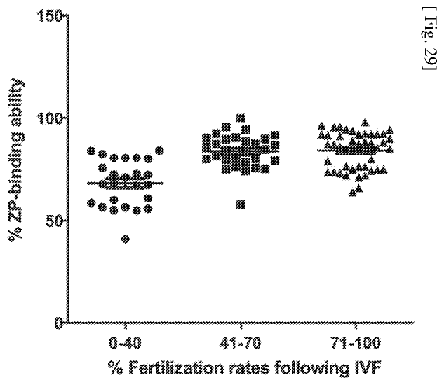

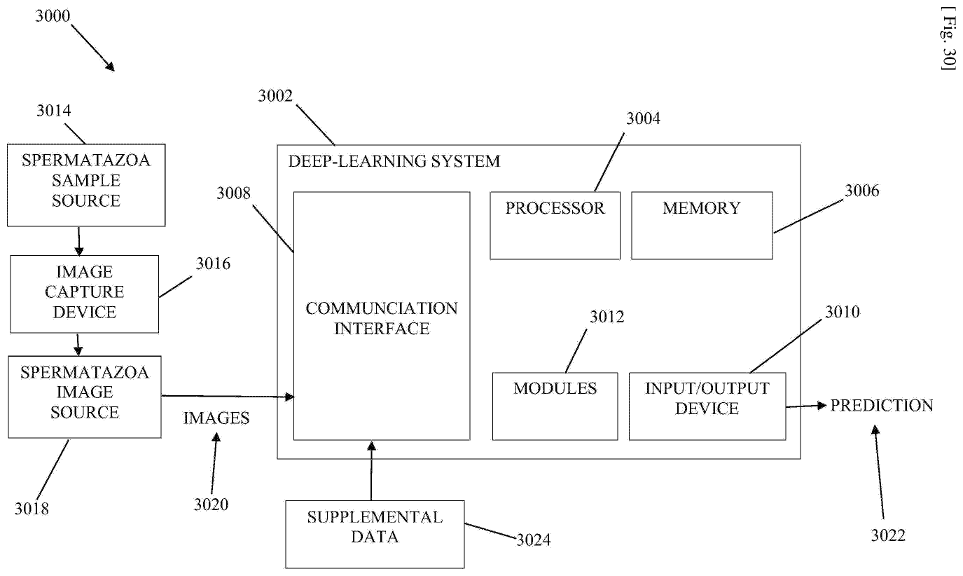

To further validate the efficacy of transfer learning for the binary classification of human spermatozoa, the classification performance was compared between a simple CNN model trained from scratch and the fine-tuned VGG-13 model. The CNN model comprised of 2 convolutional layers and 2 max pooling layers, as shown in FIG. 27A. It was trained on the datasets using a batch size of 4 over 50 epochs with a learning rate of 0.001, as shown in FIG. 27B. Although the model was able to differentiate sperm subpopulations to some extent, as shown in FIG. 27C, its overall classification performance, as shown in FIG. 27D, with accuracy: 80.4%, specificity: 81.0%, and sensitivity: 79.7%, which was inferior to the fine-tuned VGG-13 model. To further examine the generalization ability of the CNN and the fine-tuned VGG-13 model, both models were tested on an independent test dataset consisting of 122 ZP-bound spermatozoa and 98 unbound spermatozoa. The CNN model classified 68.4% of the images in the test dataset of unbound spermatozoa as ZP-unbound spermatozoa, indicating a lack of generalization power to classify unseen data, as shown in FIG. 28A. In contrast, the fine-tuned VGG-13 model successfully discriminated the two classes of spermatozoa based on their distinctive features for approximately 99% of the images, indicating excellent generalization ability, as shown in FIGS. 28A-28B.

Three groups of men (n=165) with fertilization rates of 0-40%, 41-70% and 71-100% were recruited from an assisted reproduction program to examine the relationship between ZP-binding

ability and fertilization rates of IVF using deep learning, as shown in FIG. 21. The predictive power of the model was tested on a large collection of over 40,000 images of individually extracted sperm heads from the aforementioned fertilization groups. High fertilization rate group (71-100%) had a significantly higher percentage of ZP-binding ability than the low fertilization rate group (0-40%), as in FIG. 21, with P<0.05). Further validation was conducted to compare the classification efficiency of ZP-binding ability between the model and the original VGG-13 model (without fine-tuning using the datasets). The results indicated that the original VGG-13 model lacked the capability to distinguish the ZP-bound spermatozoa from the unbound ones, as reflected by the high predicted percentages of ZP-binding ability in all fertilization groups (68.2%, 83.9%, and 84.2%), as shown in FIG. 29. The overall generalization ability of the newly fine-tuned VGG-13 model was superior to that of the original model; the AUC in discriminating the high and low fertilization rate groups was 0.97(P<0.0001 , n=113 in FIG. 22). The Youden's index was used to identify the optimal threshold value on the ROC curve generated. A cut-off value of 4.8% with a specificity of 93.8% and a sensitivity of 90.9% was established as the clinical threshold to distinguish between normal and defective ZP binding ability of the processed sperm samples.

The clinical data of 30 patients undergoing conventional insemination in IVF were randomly selected to examine the clinical significance between the conventional semen analysis and the model, as shown in FIG. 23. All patients underwent conventional IVF according to their normal semen analysis results during infertility investigation. Their semen parameters and those of the DGC-processed spermatozoa including concentration and progressive motility on the day of insemination satisfied predetermined laboratory criteria for conventional insemination. The predicted percentages of ZP-binding ability of patients 1-11 determined by the model were all above the clinical threshold of 4.8%. They all had high fertilization rates (71-100%) following conventional insemination, despite the fact that patients 2, 3, 10,and 12 had sperm morphology below the WHO reference value. On the other hand, the predicted percentages of ZP-binding ability of patients 12-21 with low fertilization rate (0-40%) were all below the threshold of the model. Among them, patients 16,17,19, 20, 21,23. 24,26, and 30 were found with suboptimal outcome despite having normal semen parameters. The findings demonstrated the prognostic value of the model highlighting the potential role of ZP-binding ability in serving as an additional and independent measure of sperm fertilizing capability, complementing the conventional semen parameters.

In an implementation consistent with the invention, as shown in FIG. 30, a system 3000 includes a deep-learning system 3002 having a hardware-based processor 3004, a memory 3006 configured to store instructions and configured to provide the instructions to the hardware-based processor 3004, a communication interface 3008, an input/output device 3010, and a set of modules 3012 configured to implement the instructions provided to the hardware-based processor 3004.

Spermatozoa samples are obtained from a spermatozoa sample source 3014, and an image capture device 3016 obtains spermatozoa images of the spermatozoa samples, as described above. The image capture device 3016 transmits, conveys, or otherwise provides the spermatozoa images to a spermatozoa image source 3018. In one implementation, the spermatozoa image source 3018 stores the captured spermatozoa images in a memory of the spermatozoa image source 3018. In another implementation, the spermatozoa image source 3018 transmits, conveys, or otherwise provides images 3020 to the deep-learning system 3002 through the communication interface 3008. For example, the images 3020 are digital still images. In another example, the images 3020 are digital still images derived from video capture of spermatozoa.

In a further implementation, the spermatozoa image source 3018 formats the images 3020 to be in a predetermined image format for processing by the deep-learning system 3002. Based on the images 3020, the deep-learning system 3002 generates a prediction 3022 regarding the percentage of spermatozoa with ZP-binding ability from the sample source 3014. The prediction 3022 is output from the input/output device 3010.

In an implementation consistent with the invention, the prediction 3022 is a binary classification. For example, the binary prediction 3022 indicates normal or defective ZP binding of the spermatozoa being evaluated. In one implementation, the predication 3022 is a message, alert, or notification displayed to a user on a display or monitor of the input/output device 3010. In another implementation, the predication 3022 is a message, alert, or notification printed out on a hardcopy by a physical printer of the input/output device 3010.

In another implementation, supplemental data 3024 is transmitted, conveyed, or otherwise provided to the deep-learning system 3002 through the communication interface 3008. For example, the supplemental data 3024 includes training data as described above for training a deep-SUBSTITUTE learning model as described herein. In another example, the supplemental data 3024 includes metadata identifying the spermatozoa and the patients providing the spermatozoa.

In one implementation, the deep-learning system 3002 is operatively connected to data sources such as the spermatozoa image source 3018 and the supplemental data through a network. For example, the network is the Internet. In another example, the network is an internal network or intranet of an organization. In a further example, the network is a heterogeneous or hybrid network including the Internet and the intranet.

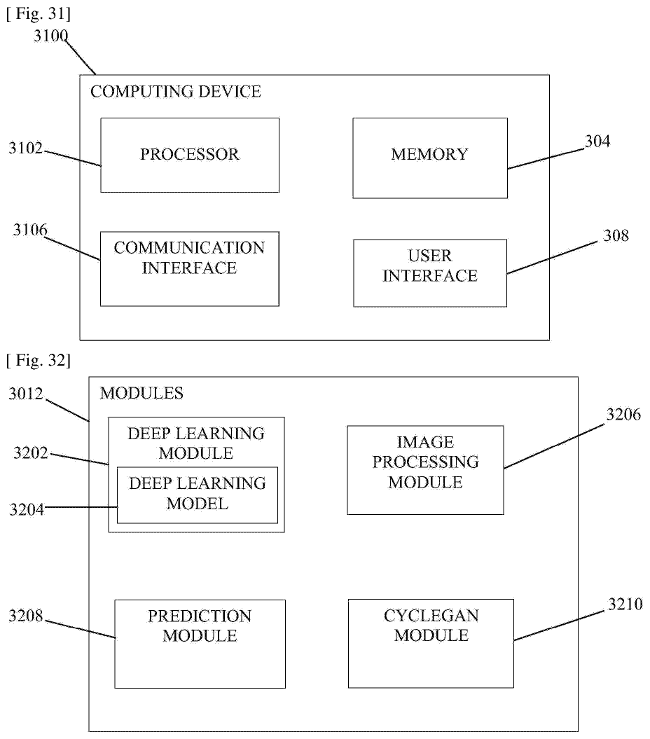

FIG. 31 illustrates a schematic of a computing device 3100 including a processor 3102 having code therein, a memory 3104, and a communication interface 3106. Optionally, the computing device 3100 can include a user interface 3108, such as an input device, an output device, or an input/output device, such as the input/output device 3010. The processor 3102, the memory 3104, the communication interface 3106, and the user interface 3108 are operatively connected to each other via any known connections, such as a system bus, a network, etc. Any component, combination of components, and modules of the system 3000 in FIG. 30 can be implemented by a respective computing device 3100. For example, each of the components 3002-3018 shown in FIG. 30 can be implemented by a respective computing device 3100 shown in FIG. 31 and described below.

It is to be understood that the computing device 3100 can include different components. Alternatively, the computing device 3100 can include additional components. In another alternative implementation, some or all of the functions of a given component can instead be carried out by one or more different components. The computing device 3100 can be implemented by a virtual computing device. Alternatively, the computing device 3100 can be implemented by one or more computing resources in a cloud computing environment. Additionally, the computing device 3100 can be implemented by a plurality of any known computing devices.

The processor 3102 can be a hardware-based processor implementing a system, a sub-system, or a module. The processor 3102 can include one or more general-purpose processors. Alternatively, the processor 3102 can include one or more special-purpose processors. The processor 3102 can be integrated in whole or in part with the memory 3104, the communication interface 3106, and the user interface 3108. In another alternative implementation, the processor 3102 can be implemented by any known hardware-based processing device such as a controller, an integrated circuit, a microchip, a central processing unit (CPU), a microprocessor, a system on a chip (SoC), a field-programmable gate array (FPGA), or an application-specific integrated circuit (ASIC). In addition, the processor 3102 can include a plurality of processing elements configured to perform parallel processing. In a further alternative implementation, the processor 3102 can include a plurality of nodes or artificial neurons configured as an artificial neural network. The processor 3102 can be configured to implement any known machine learning (ML) based devices, any known artificial intelligence (AI) based devices, and any known artificial neural networks, including a convolutional neural network (CNN).

The memory 3104 can be implemented as a non-transitory computer-readable storage medium such as a hard drive, a solid-state drive, an erasable programmable read-only memory (EPROM), a universal serial bus (USB) storage device, a floppy disk, a compact disc read-only memory (CD-ROM) disk, a digital versatile disc (DVD), cloud-based storage, or any known nonvolatile storage.

The code of the processor 3102 can be stored in a memory internal to the processor 3102. The code can be instructions implemented in hardware. Alternatively, the code can be instructions implemented in software. The instructions can be machine-language instructions executable by the processor 3102 to cause the computing device 3100 to perform the functions of the computing device 3100 described herein. Alternatively, the instructions can include script instructions executable by a script interpreter configured to cause the processor 3102 and computing device 3100 to execute the instructions specified in the script instructions. In another alternative implementation, the instructions are executable by the processor 3102 to cause the computing device 3100 to execute an artificial neural network. The processor 3102 can be implemented using hardware or software, such as the code. The processor 3102 can implement a system, a sub-system, or a module, as described herein.

The memory 3104 can store data in any known format, such as databases, data structures, data lakes, or network parameters of a neural network. The data can be stored in a table, a flat file, data in a filesystem, a heap file, a B+tree, a hash table, or a hash bucket. The memory 3104 can be implemented by any known memory, including random access memory (RAM), cache memory, register memory, or any other known memory device configured to store instructions or data for rapid access by the processor 3102, including storage of instructions during execution.

The communication interface 3106 can be any known device configured to perform the communication interface functions of the computing device 3100 described herein. The communication interface 3106 can implement wired communication between the computing device 3100 and another entity. Alternatively, the communication interface 3106 can implement wireless communication between the computing device 3100 and another entity. The communication interface 3106 can be implemented by an Ethernet, Wi-Fi, Bluetooth, or USB interface. The communication interface 3106 can transmit and receive data over a network and to other devices using any known communication link or communication protocol.

The user interface 3108 can be any known device configured to perform user input and output functions. The user interface 3108 can be configured to receive an input from a user. Alternatively, the user interface 3108 can be configured to output information to the user. The user interface 3108 can be a computer monitor, a television, a loudspeaker, a computer speaker, or any other known device operatively connected to the computing device 3100 and configured to output information to the user. A user input can be received through the user interface 3108 implementing a keyboard, a mouse, or any other known device operatively connected to the computing device 3100 to input information from the user. Alternatively, the user interface 3108 can be implemented by any known touchscreen. The computing device 3100 can include a server, a personal computer, a laptop, a smartphone, or a tablet.

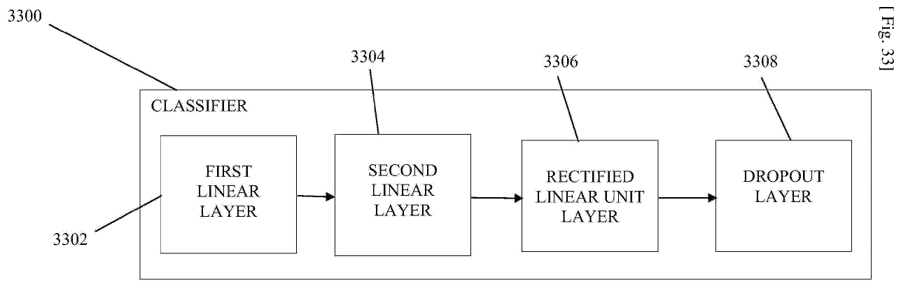

As shown in FIG. 32, the modules 3012 used in the implementation of the system 3000 of FIG. 30 include at least a deep learning module 3202 configured to operate a deep learning model 3204 such as VGG-13. The deep learning model 3204 is trained and configured as described herein to generate the prediction 3022 from the images 3020. The modules 3012 also include an image processing module 3206, a prediction module 3208, and a CycleGAN module 3210. The image processing module 3206 is configured to process digital images 3020 as described above. The prediction module 3208 is configured to predict in vitro fertilization (IVF) success of spermatozoa based on their ZP-binding ability, such that sperm samples with normal ZP-binding ability predict successful IVF, and those sperm samples with defective ZP-binding ability predict unsuccessful IVF. The CycleGAN module 3210 is set up to apply CycleGAN processing to digital images 3020 of ZP-unbound, using the microenvironment of ZP-bound spermatozoa as a reference. These images 3020 are then inputted into the deep learning model 3204.

In an implementation, the deep learning module 3202 is configured as a CNN including feature extractor and a classifier, as described above with regards to FIGS. 9, 12, 18, and 27. In an alternative implementation, shown in FIG. 33, the classifier 3300 is configured to have a first linear layer 3302, a second linear layer 3304, a rectified linear unit (ReLU) layer 3306, and a dropout layer 3308. The configuration of the classifier 3300 is finetuned for the specific downstream task of sperm classification while leveraging the capability of the deep learning model 3204, such as a pretrained VGG-13 model, as described above.

Referring to FIG. 34, a method 3400 is provided to collect spermatozoa to be the spermatozoa sample source 3014 in FIG. 30, and to capture images 3020 of the collected spermatozoa stored in the spermatozoa image source 3018 in FIG. 30. As shown in FIG. 34, the method 3400 includes collecting spermatozoa and oocyte samples in step 3402, selecting normospermic samples according to a predetermined WHO criteria in step 3404 and as described above, and isolating motile and viable spermatozoa from the seminal plasma in step 3406. The method 3400 then, in step 3408, collects ZP-bound spermatozoa by incubating the processed spermatozoa in a culture medium with a group of human oocytes to isolate ZP-bound spermatozoa within 30 min., and then in step 3410, collects ZP-bound spermatozoa from clinical samples with defective ZP-binding ability, as evidenced by complete fertilization failure following conventional IVF and the absence of ZP-bound spermatozoa on the inseminated oocytes. The collected spermatozoa are then stained using diff-quik in step 3412, as described above, and images 3020 of the stained spermatozoa are captured in step 3414 through one or more imaging modalities. For example, the images 3020 are captured with oil immersion under a light microscope (Zeiss, Gottingen, Germany), as the image capture device 3016 in FIG. 30, at a magnification of 1000×, as described above. In another example, the images are captured with any known camera device or any known imaging device as the image capture device 3016.

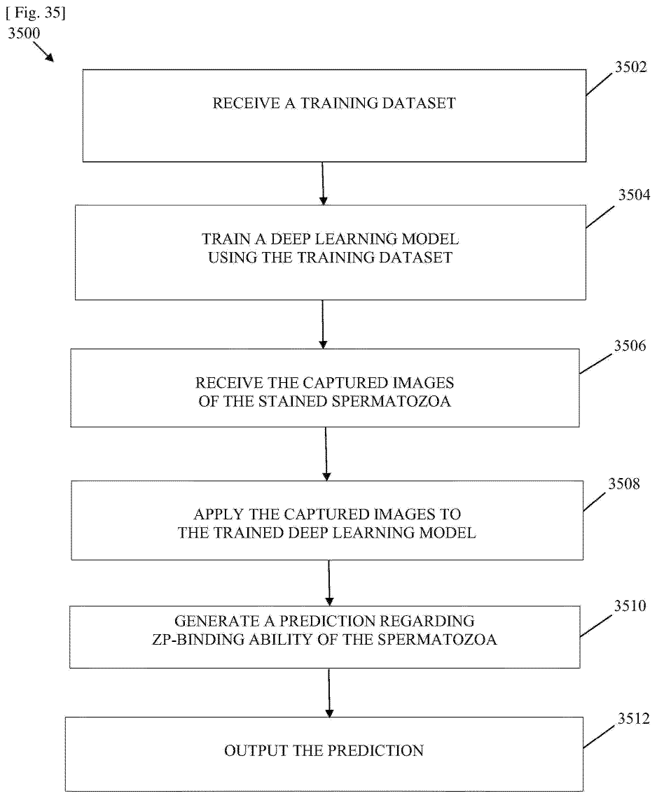

In an implementation consistent with the invention, as shown in FIG. 35, the method 3500 of operation of the system 3000 shown in FIG. 30 includes the steps of receiving a training dataset in step 3502, such as training data included in the supplemental data 3024, and the deep learning model 3204 executed by the deep learning module 3202 is trained using the training dataset in step 3504. The training dataset includes previously obtained images as described above. Once the deep learning model 3204 is trained, the method 3500 receives the captured images 3020 of the stained spermatozoa in step 3506, applies the captured images 3020 to the trained deep learning model 3204 in step 3508, generates a prediction 3022 in step 3510 regarding the ZP-binding ability of the spermatozoa represented by the images 3020, and outputs the prediction 3022 in step 3512 using the input/output device 3010.

With the trained model, deep learning can be used to analyze sperm head images based on their morphological characteristics to predict the fertilization potential of human spermatozoa. The fine-tuned deep learning model exhibited a good generalization ability to classify sperm subpopulations with high discriminative power. The predictive threshold established by clinical data can be employed to assess the ZP-binding ability of clinical specimens for predicting fertilization outcome following conventional insemination.

Although conventional semen analysis is commonly used to examine sperm parameters in male infertility investigation, they are surrogate markers of fertility which cannot fully reflect the fertilization potential of spermatozoa. Men with normal semen parameters can be infertile because they can have spermatozoa with functional defects undetectable by the surrogate parameters, leading to unexpected fertilization failure in IVF with conventional insemination. On the other hand, men with semen parameters below the WHO reference values are not necessarily infertile because the reference values are derived from fertile men. Moreover, semen parameters vary greatly among individuals, making it difficult to establish definitive thresholds for prediction of fertilization outcome. Consequently, the clinical significance of the WHO reference values of semen parameters remains controversial. Furthermore, manual semen analysis relies heavily on technicians' experience and judgement, which can result in significant intra-and inter-assay variations due to the subjective nature of the assessment methods. Despite major technological advancement, fertilization failure occurs in 5-10% of IVF cycles with conventional insemination. A recent large retrospective cohort study concluded that the predictive power of semen parameters for total fertilization failure following conventional insemination in IVF was limited.

Deep learning has emerged as a popular approach for medical image analysis because of its high versatility and reliability. The method has been used to identify abnormalities in abdominal ultrasound images and risk factors of cardiovascular diseases. Lately, there has been a surge of interest in exploring the use of deep learning for sperm morphology evaluation. However, these models were trained with datasets annotated by embryologists according to the WHO strict criteria, which could introduce bias and subjectivity in the training process. Ideally, deep learning should be inherently capable of learning from the input data with minimal human intervention.

Despite being a vital element of conventional semen analysis, conflicting results have been reported on the limited predictive power of sperm morphology for sperm quality, and fertilization outcomes in assisted reproduction treatments. In contrast, ZP-binding ability of spermatozoa is an independent, surrogate matric of sperm quality closely related to high rates of acrosome reaction and DNA integrity, and positively correlated with fertilization rates following conventional insemination in IVF. Hemizona assay is developed to evaluate the ZP-binding ability of spermatozoa by incubation of matching equally bisected hemizona with samples from fertile men and patients for clinical diagnosis. However, the need for micromanipulation to bisect the ZP and limited availability of human oocytes restrict a wide application of the method for routine use.

The present disclosure describes a novel method to identify spermatozoa with/without ZP-binding ability according to their morphological features using an advanced deep learning model. During the method development stage, a representative database containing Diff-Quik stained images of ZP-bound spermatozoa and spermatozoa with defective ZP-binding ability was established, which could be accessible to the public in the future for clinical uses. Individual sperm heads up to the neckpieces were extracted from the collected images using K-means clustering algorithm, which identified specific targets of interest from the background by grouping images with a vector of pixel values similar to a reference image. The clinical implications of the morphological features of the sperm tails were much more complex compared to those of the sperm heads, mainly due to lack of standardized criteria for sperm tail morphology assessment and high variations among individual spermatozoa within a single sample. It has been suggested that the presence of sperm tail within the visual range led to biased, unreliable predictions of DNA quality in relation to sperm head morphology using deep learning.

ZP-bound spermatozoa were collected by co-incubating swim-up processed spermatozoa with the oocytes, and unbound spermatozoa were the residual DGC-processed clinical samples with total fertilization failure following conventional insemination in IVF. Considering the potential difference in the microenvironment between these samples, CycleGAN was used to translate the specific characteristics of the processed ZP-bound spermatozoa into the clinical sperm samples to generate a more consistent dataset representative of sperm morphology. CycleGAN is a powerful type of GAN designed to transfer acquired knowledge between two different image domains for image-to-image translation. CycleGAN consists of two generators and two discriminators to produce plausible and high-quality images by training in opposition to each other. To be more specific, the generators create converted images in one domain, which is evaluated by the discriminators for authenticity, mainly for determination of how convincing the converted images appear when compared to the reference images from the target domain.

Training a deep learning model from scratch with the ability of generalization to unseen data requires a substantial amount of training data and computational power. Transfer learning is commonly used to fine-tune the classifier of a pre-trained model for a new task by retraining the final layers of the model on a small dataset while retaining all the optimized weights acquired from a large, pre-existing dataset. This approach is particularly useful in medical image analysis to address the limitations of sample availability and manual annotations, which can significantly improve classification accuracy and training efficiency without overfitting. Recently, it has been used to fine-tune two pre-trained deep learning models for sperm segmentation to automatically identify and label the sperm head, acrosome, and nucleus, with an average agreement of 95% with the manually segmented regions. Another study further explore the use of this technique to classify sperm morphology according to the WHO criteria with an accuracy of 96.0% and a precision of 96.4%.

Considering the limited availability of ZP-bound and unbound spermatozoa, VGG-13 model was used as a feature extractor that had been previously trained on a diverse ImageNet database to extract relevant and distinctive features of input images, which are used then as training data to develop a new classifier during fine-tuning for the specific task of classifying ZP-bound and unbound spermatozoa with high specificity and sensitivity. A classic, simple CNN model was also trained from scratch to further examine the advantages of transfer learning on the limited datasets. Although the CNN model demonstrated reasonable classification performance, it lacked the ability to effectively generalize to unseen clinical data that were derived from the same condition as the training set. One possible explanation is that the model failed to extract unique and distinctive features from the small datasets to establish specific patterns of representation due to the high morphological variations among individual spermatozoa within each class.

Deep learning models do not generally provide comprehensive information regarding the decision-making process owing to their uninterpretable “black box” nature. Saliency map is one method to visualize and conceptually interpret the decision-making process by revealing the pixels localized to the specific region(s) of an image that contribute to the classification process. This technique has been used to validate the effectiveness of deep learning to identify the regions of interest, such as abnormalities and cell types in medical images. The heat maps generated using saliency maps indicated that the model predominantly detected sperm head and mid-piece, while disregarding the background noises and debris. Previous results reported that the ZP-binding ability of spermatozoa were significantly associated with higher rates of normal morphology. However, these studies did not identify the most important morphological features associated with the ZP-binding ability of spermatozoa. The model revealed that the anterior region pixels is a major morphological factor contributing to its learning and classification processes, consistent with the biological significance of the sperm acrosomal region for ZP-binding. Through the use of transfer learning and data argumentation, the relevant features are extracted from the datasets to establish hidden yet interconnected patterns of each class for binary classification, by leveraging the training weights and hyperparameters of the pre-trained VGG-13 model.

To translate these findings into clinical practice, sperm samples with different fertilization rates following conventional insemination in IVF were collected to validate the discriminatory power of the pre-trained VGG-13 model for ZP-binding ability of human spermatozoa. The results demonstrated that the total percentage of ZP-binding ability varied distinctively among different fertilization rate groups. The high fertilization rate group (71-100%) had a significantly higher percentage of ZP-binding ability than the low fertilization rate group (0%-40%), consistent with previous report on differences in the number of motile spermatozoa with ZP-binding ability between fertile and infertile men. Considering the high predictive power of ZP-binding ability for fertilization outcome following conventional insemination in IVF, the data generated by the model was used to determine the threshold to distinguish between normal and defective ZP-binding ability of spermatozoa. When compared with the conventional semen results alone, the model provided additional information on sperm fertilization potential irrespective of the sperm parameters. The model enables identification of men who are likely to fail in IVF using conventional insemination due to defective ZP-binding ability despite having normal semen parameters in semen analysis. The information can allow patients to make informed decisions on the fertilization method (conventional insemination vs. ICSI) to be used, preventing psychological and financial sufferings to the patients caused by fertilization failure.

The model is applicable to Diff-Quik stained images captured at high resolution. Its completability with the other staining methods remains to be optimized. It could also be utilized for non-stained images in a comparable manner. A deep learning model has been established to detect morphologically abnormal spermatozoa from non-stained and relatively low-resolution images. Known deep learning-based models have been trained on stain-free spermatozoa of known DNA quality for predictions with brightfield images. The disclosed model can be tested on datasets of semen samples collected under different preparation conditions, such as variations in sample processing method and staining protocol, to ensure consistent classification accuracy. The disclosed model can serve as a foundation for developing an advanced platform that combines microfluidics and deep learning to simultaneously identify and separate high-quality, viable sperm cells for ICSI. Microfluidics has been used to isolate specific sperm subpopulations based on their biological parameters and physiological properties. Using a high-resolution, time-lapse imaging system, the integrated platform can instantly examine and select freely moving sperm based on their morphological features through deep learning. Recent researches has successfully employed deep learning-based approaches to analyze video recordings of sperm motility, showcasing the potential of this technology for live sperm selection.

The disclosed model can evaluate the fertilization potential of the Diff-Quik stained spermatozoa according to their morphological features using deep learning. Patients with a predicted percentage of ZP-binding ability below 4.8% should consider undergoing ICSI instead of conventional insemination to improve the fertilization outcome. The classification performance of the model can be further improved by increasing the sample sizes of semen samples captured under various platforms so as to enhance its sensitivity and specificity with varying levels of image quality, allowing the use of low-cost, readily available equipment for analysis.

Accordingly, the disclosed fine-tuned model incorporating ZP-binding ability allows 1) real-time evaluation of sperm fertilizing potential independent of those derived from the conventional semen analysis; and 2) identification of patients with defective ZP-binding ability by Diff-Quik stained image analysis using deep learning. In particular, the threshold for morphological assessment can be used to identify men whose spermatozoa possess inferior fertilizing ability, which may lead to a high likelihood of fertilization failure in conventional insemination in IVF. The development of a reliable and robust method for sperm evaluation allows informed decision-making between ICSI and conventional insemination, potentially leading to improved fertilization results. Ultimately, this can contribute to a better clinical management of infertile couples undergoing assisted reproduction treatments.

### Exemplary Products, Systems and Methods Are Set out in the Following Items

1. A deep learning-based method for assisted reproduction comprising:
a. obtaining at least one sample of zona pellucida (ZP)-bound spermatozoa and at least one sample of ZP-unbound spermatozoa;
b. obtaining one or more digital images of said (ZP)-bound and ZP-unbound spermatozoa through one or more imaging modalities; and
c. processing the one or more digital images according to at least one deep learning model configured to use sperm images to generate binary prediction regarding ZP-binding ability of spermatozoa, wherein the binary prediction is normal or defective ZP binding.
2. The method of any preceding item, wherein the at least one deep learning model is transfer learning.
3. The method of any preceding item, wherein the at least one deep learning model is selected from the group consisting of Visual Geometry Group (VGG), LeNet, AlexNet, U Net, and Residual Neural Network (ResNet).
4. The method of any preceding item, wherein the deep learning model is VGG-13.
5. The method of any preceding item, further comprising the step of processing said digital images by an unsupervised machine learning clustering algorithm.
6. The method of any preceding item, wherein the unsupervised machine learning algorithm is selected from the group consisting of a k-means clustering algorithm, a gaussian mixture model, hierarchical clustering, spectral clustering, and k-medoids clustering.
7. The method of any preceding item, further comprising the step of staining the at least one sample of ZP-bound spermatozoa and the at least one sample of ZP-unbound spermatozoa prior to step b.
8. The method of any preceding item, further comprising performing cycle generative adversarial network (CycleGAN) to said digital images of ZP-unbound and ZP-bound spermatozoa prior to step c.
9. The method of any preceding item, wherein the threshold value to distinguish between normal and defective ZP-binding ability of spermatozoa is about 4.78%.
10. The method of any preceding item, wherein the ZP-unbound spermatozoa were obtained from clinical samples with defective ZP-binding ability, wherein said clinical samples failed conventional insemination during IVF.
11. The method of any preceding item, wherein the ZP-bound spermatozoa were obtained by ZP-spermatozoa coincubation wherein the motile spermatozoa were incubated with one or more oocytes for 30 minutes to retrieve spermatozoa capable of binding to the ZP.
12. The method of any preceding item, wherein said deep learning model VGG-13 predicts ZP-binding ability with at least about 90 % accuracy, specificity, sensitivity, or a combination thereof.
13. The method of any preceding item, wherein the ZP-binding ability is predicted with specificity or sensitivity of at least about 90%.
14. The method of any preceding item, wherein the ZP-binding ability is predicted with specificity or sensitivity of at least about 95%.
15. The method of any preceding item, further comprising the step of predicting in vitro fertilization (IVF) success of spermatozoa based on their ZP-binding ability, wherein sperm samples with normal ZP-binding ability predict successful IVF, and those with defective ZP-binding ability predict unsuccessful IVF.

16. A method of predicting fertilization success following conventional insemination during IVF, comprising:
a. obtaining a sperm sample from a subject undergoing IVF;
b . obtaining one or more digital images of said sperm sample through one or more imaging modalities; and
c. processing the one or more digital images according to at least one deep learning model according to item 1 to obtain a binary prediction regarding ZP-binding ability of said sperm sample, wherein normal ZP-binding of said sperm sample predicts fertilization success, and defective ZP-binding of said sperm sample predicts fertilization failure.
17. The method of any preceding item, wherein the at least one deep learning model is transfer learning.
18. The method of any preceding item, wherein the at least one deep learning model is selected from the group consisting of Visual Geometry Group (VGG), LeNet, AlexNet, U Net, and Residual Neural Network (ResNet).
19. The method of any preceding item, wherein the deep learning model is VGG-13.
20. The method of any preceding item, further comprising the step of processing said digital images by an unsupervised machine learning clustering algorithm.
21. The method of any preceding item, wherein the unsupervised machine learning algorithm is selected from the group consisting of a k-means clustering algorithm, a gaussian mixture model, hierarchical clustering, spectral clustering, and k-medoids clustering.
22. The method of any preceding item, further comprising the step of staining the sperm sample prior to step b.
23. The method of any preceding item, wherein the threshold value to distinguish between normal and defective ZP-binding ability of the test sperm sample is about 4.78% ZP-binding.
24. The method of any preceding item, wherein said VGG-13 model predicts ZP-binding ability with at least 90% accuracy, specificity, sensitivity, or a combination thereof.
25. The method of any preceding item, wherein the ZP-binding ability is predicted with a specificity or sensitivity of at least about 90%.
26. The method of any preceding item, wherein the ZP-binding ability is predicted with a specificity or sensitivity of at least about 95%.
27. A method for predicting the ZP-binding ability of spermatozoa, comprising:
a. obtaining at least one sample of ZP-bound spermatozoa and at least one sample of ZP-unbound spermatozoa;
b. obtaining one or more digital images of said ZP-bound and ZP-unbound spermatozoa through one or more imaging modalities; and
c. processing the one or more digital images according to at least one deep learning model configured to use sperm images to generate a binary prediction regarding ZP-binding ability of spermatozoa, wherein the binary prediction is normal or defective ZP binding; wherein said deep learning model is VGG-13 which predicts ZP-binding ability with at least about 90 % accuracy, specificity, sensitivity, or a combination thereof.
28. A deep learning-based system for assisted reproduction comprising:
   a. means for obtaining at least one sample of zona pellucida (ZP)-bound spermatozoa and at least one sample of ZP-unbound spermatozoa;
   b. means for obtaining one or more digital images of said (ZP)-bound and ZP-unbound spermatozoa through one or more imaging modalities;
   c. a hardware-based processor;
   d. a memory configured to store instructions and configured to provide the instructions to the hardware-based processor; and
   e. a set of modules configured to implement the instructions provided to the hardware-based processor, the set of modules including:
      a deep learning module configured to implement at least one deep learning model, to process the one or more digital images, and to use sperm images to generate a binary prediction regarding ZP-binding ability of spermatozoa, wherein the binary prediction is normal or defective ZP binding.
29. The system of any preceding item, wherein the at least one deep learning model is transfer learning.
30. The system of any preceding item, wherein the at least one deep learning model is selected from the group consisting of Visual Geometry Group (VGG), LeNet, AlexNet, U Net, and Residual Neural Network (ResNet).
31. The system of any preceding item, wherein the at least one deep learning model is VGG-13.
32. The system of any preceding item, further comprising a processing module configured to process said digital images by an unsupervised machine learning clustering algorithm.
33. The system of any preceding item, wherein the unsupervised machine learning algorithm is selected from the group consisting of a k-means clustering algorithm, a gaussian mixture model, hierarchical clustering, spectral clustering, and k-medoids clustering.
34. The system of any preceding item, further comprising means for staining the at least one sample of ZP-bound spermatozoa and the at least one sample of ZP-unbound spermatozoa prior to obtaining the one or more digital images using the means for obtaining one or more digital images.
35. The system of any preceding item, further comprising a cycle generative adversarial network (CycleGAN) module configured to perform CycleGAN to said digital images of ZP-unbound and ZP-bound spermatozoa prior to implementing the at least one deep learning model.
36. The system of any preceding item, wherein the threshold value to distinguish between normal and defective ZP-binding ability of spermatozoa is about 4.78%.
37. The system of any preceding item, wherein the ZP-unbound spermatozoa were obtained from clinical samples with defective ZP-binding ability, wherein said clinical samples failed conventional insemination during IVF.
38. The system of any preceding item, wherein the ZP-bound spermatozoa were obtained by ZP-spermatozoa coincubation wherein the motile spermatozoa were incubated with one or more oocytes for 30 minutes to retrieve spermatozoa capable of binding to the ZP.
39. The system of any preceding item, wherein said deep learning model VGG-13 predicts ZP-binding ability with at least about 90 % accuracy, specificity, sensitivity, or a combination thereof.
40. The system of any preceding item, wherein the ZP-binding ability is predicted with specificity or sensitivity of at least about 90%.
41. The system of any preceding item, wherein the ZP-binding ability is predicted with specificity or sensitivity of at least about 95%.
42. The system of any preceding item, further comprising a prediction module configured to predict in vitro fertilization (IVF) success of spermatozoa based on their ZP-binding ability, wherein sperm samples with normal ZP-binding ability predict successful IVF, and those with defective ZP-binding ability predict unsuccessful IVF.
43. A system of predicting fertilization success following conventional insemination during IVF, comprising:
   a. means for obtaining a sperm sample from a subject undergoing IVF;
   b. means for obtaining one or more digital images of said sperm sample through one or more imaging modalities; and
   c. the deep learning-based system of item 28 configured to obtain a binary prediction regarding ZP-binding ability of said sperm sample, wherein normal ZP-binding of said sperm sample predicts fertilization success, and defective ZP-binding of said sperm sample predicts fertilization failure.
44. The system of any preceding item, wherein the at least one deep learning model is transfer learning.
45. The system of any preceding item, wherein the at least one deep learning model is selected from the group consisting of Visual Geometry Group (VGG), LeNet, AlexNet, U Net, and Residual Neural Network (ResNet).
46. The system of any preceding item, wherein the deep learning model is VGG-13.
47. The system of any preceding item, further comprising a processing module configured to process said digital images by an unsupervised machine learning clustering algorithm.
48. The system of any preceding item, wherein the unsupervised machine learning algorithm is selected from the group consisting of a k-means clustering algorithm, a gaussian mixture model, hierarchical clustering, spectral clustering, and k-medoids clustering.
49. The system of any preceding item, further comprising means for staining the sperm sample prior to obtaining the one or more digital images using the means for obtaining one or more digital images.
50. The system of any preceding item, wherein the threshold value to distinguish between normal and defective ZP-binding ability of the test sperm sample is about 4.78% ZP-binding.
51. The method of any preceding item, wherein said VGG-13 model predicts ZP-binding ability with at least 90% accuracy, specificity, sensitivity, or a combination thereof.
52. The system of any preceding item, wherein the ZP-binding ability is predicted with a specificity or sensitivity of at least about 90%.
53. The system of any preceding item, wherein the ZP-binding ability is predicted with a specificity or sensitivity of at least about 95%.
54. A system for predicting the ZP-binding ability of spermatozoa, comprising:
   a. means for obtaining at least one sample of ZP-bound spermatozoa and at least one sample of ZP-unbound spermatozoa;
   b. means for obtaining one or more digital images of said ZP-bound and ZP-unbound spermatozoa through one or more imaging modalities; and
   c. a hardware-based processor;
   d. a memory configured to store instructions and configured to provide the instructions to the hardware-based processor; and
   e. a set of modules configured to implement the instructions provided to the hardware-based processor, the set of modules including:
      a deep learning module configured to implement at least one deep learning model, to process the one or more digital images according to at least one deep learning model configured to use sperm images, and to generate a binary prediction regarding ZP-binding ability of spermatozoa, wherein the binary prediction is normal or defective ZP binding;
   wherein said deep learning model is VGG-13 which predicts ZP-binding ability with at least about 90 % accuracy, specificity, sensitivity, or a combination thereof.
55. The system of any preceding item, further comprising:
   a classifier including, in sequence:
      a first linear layer;
      a second linear layer;
      a rectified linear unit (ReLU) layer; and
      a dropout layer.
56. The method of any preceding item, further comprising, prior to step b, the steps of:
   selecting normospermic samples from the obtained samples of spermatozoa according to a predetermined criteria;
   isolating motile and viable spermatozoa from seminal plasma;
   incubating the isolated spermatozoa in a predetermined solution; and
   staining the incubated spermatozoa.
57. The method of any preceding item, further comprising, prior to step c:
   receiving a training dataset; and
   training the at least one deep learning model using the training dataset.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The terms “computer readable medium” and “machine readable medium” includes, but is not limited to portable or fixed storage devices, optical storage devices, wireless channels, and/or various other storage mediums capable of storing that contain or carry instruction(s) and/or data. It will be appreciated that a “computer readable medium” or “machine readable medium” can include multiple media each operatively connected to a processing unit.

### REFERENCES

1 The Annual Scientific Meeting of Hong Kong Society of Endocrinology, Metabolism and Reproduction, Hong Kong. (Oral and poster presentation)
2. Deep Learning for the classification of human spermatozoa.

1. A deep learning-based method for assisted reproduction comprising:
a. obtaining at least one sample of zona pellucida (ZP)-bound spermatozoa and at least one sample of ZP-unbound spermatozoa;
b. obtaining one or more digital images of said (ZP)-bound and ZP-unbound spermatozoa through one or more imaging modalities; and
c. processing the one or more digital images according to at least one deep learning model configured to use the one or more digital images to generate a binary prediction regarding ZP-binding ability of spermatozoa, wherein the binary prediction is normal or defective ZP binding.

2. The method of claim 1, wherein the at least one deep learning model is transfer learning.

3. The method of claim 1, wherein the at least one deep learning model is selected from the group consisting of Visual Geometry Group (VGG), LeNet, AlexNet, U Net, and Residual Neural Network (ResNet).

4. The method of claim 1, wherein the deep learning model is VGG-13.

5. The method of any claim 1, further comprising the step of processing said digital images by an unsupervised machine learning clustering algorithm.

6. The method of claim 1, wherein the unsupervised machine learning algorithm is selected from the group consisting of a k-means clustering algorithm, a gaussian mixture model, hierarchical clustering, spectral clustering, and k-medoids clustering.

7. The method of claim 1, further comprising the step of staining the at least one sample of ZP-bound spermatozoa and the at least one sample of ZP-unbound spermatozoa prior to step b.

8. The method of claim 1, further comprising performing cycle generative adversarial network (CycleGAN) to said digital images of ZP-unbound and ZP-bound spermatozoa prior to step c.

9. The method of claim 1, wherein the threshold value to distinguish between normal and defective ZP-binding ability of spermatozoa is about 4.78%.

10. The method of claim 1, wherein the ZP-unbound spermatozoa were obtained from clinical samples with defective ZP-binding ability, wherein said clinical samples failed conventional insemination during-IVF in vitro fertilization (IVF).

11. The method of claim 1, wherein the ZP-bound spermatozoa were obtained by ZP-spermatozoa coincubation wherein the motile spermatozoa were incubated with one or more oocytes for 30 minutes to retrieve spermatozoa capable of binding to the ZP.

12. The method of claim 1, wherein said deep learning model VGG-13 predicts ZP-binding ability with at least about 90 % accuracy, specificity, sensitivity, or a combination thereof.

13. The method of claim 1, wherein the ZP-binding ability is predicted with specificity or sensitivity of at least about 90%.

14. The method of claim 1, wherein the ZP-binding ability is predicted with specificity or sensitivity of at least about 95%.

15. The method of claim 1, further comprising the step of predicting in vitro fertilization (IVF) success of spermatozoa based on their ZP-binding ability, wherein sperm samples with normal ZP-binding ability predict successful IVF, and those with defective ZP-binding ability predict unsuccessful IVF.

16. A method of predicting fertilization success following conventional insemination during in vitro fertilization (IVF), comprising:
a. obtaining a sperm sample from a subject undergoing IVF;
b. obtaining one or more digital images of said sperm sample through one or more imaging modalities; and
c. processing the one or more digital images of said sperm sample according to at least one deep learning model according to claim 1 to obtain a binary prediction regarding ZP-binding ability of said sperm sample, wherein normal ZP-binding of said sperm sample predicts fertilization success, and defective ZP-binding of said sperm sample predicts fertilization failure.

17. The method of claim 1, wherein the at least one deep learning model is transfer learning.

18. The method of claim 1, wherein the at least one deep learning model is selected from the group consisting of Visual Geometry Group (VGG), LeNet, AlexNet, U Net, and Residual Neural Network (ResNet).

19. The method of claim 1, wherein the deep learning model is VGG-13.

20. The method of claim 1, further comprising the step of processing said digital images by an unsupervised machine learning clustering algorithm.

21. The method of claim 1, wherein the unsupervised machine learning algorithm is selected from the group consisting of a k-means clustering algorithm, a gaussian mixture model, hierarchical clustering, spectral clustering, and k-medoids clustering.

22. The method of claim 1, further comprising the step of staining the sperm sample prior to step b.

23. The method of claim 1, wherein the threshold value to distinguish between normal and defective ZP-binding ability of the test sperm sample is about 4.78% ZP-binding.

24. The method of claim 1, wherein said VGG-13 model predicts ZP-binding ability with at least 90% accuracy, specificity, sensitivity, or a combination thereof.

25. The method of claim 1, wherein the ZP-binding ability is predicted with a specificity or sensitivity of at least about 90%.

26. The method of claim 1, wherein the ZP-binding ability is predicted with a specificity or sensitivity of at least about 95%.

27. A method for predicting the ZP-binding ability of spermatozoa, comprising:
a. obtaining at least one sample of ZP-bound spermatozoa and at least one sample of ZP-unbound spermatozoa;
b. obtaining one or more digital images of said ZP-bound and ZP-unbound spermatozoa through one or more imaging modalities; and
c. processing the one or more digital images according to at least one deep learning model configured to use the one or more digital images to generate a binary prediction regarding ZP-binding ability of spermatozoa, wherein the binary prediction is normal or defective ZP binding;
wherein said deep learning model is VGG-13 which predicts ZP-binding ability with at least about 90 % accuracy, specificity, sensitivity, or a combination thereof.

28. A deep learning-based system for assisted reproduction comprising:
a. means for obtaining at least one sample of zona pellucida (ZP)-bound spermatozoa and at least one sample of ZP-unbound spermatozoa;
b. means for obtaining one or more digital images of said (ZP)-bound and ZP-unbound spermatozoa through one or more imaging modalities;
c. a hardware-based processor;
d. a memory configured to store instructions and configured to provide the instructions to the hardware-based processor; and
e. a set of modules configured to implement the instructions provided to the hardware-based processor, the set of modules including:
a deep learning module configured to implement at least one deep learning model, to process the one or more digital images, and to use the one or more digital images to generate a binary prediction regarding ZP-binding ability of spermatozoa, wherein the binary prediction is normal or defective ZP binding.

29. The system of claim 28, wherein the at least one deep learning model is transfer learning.

30. The system of claim 28, wherein the at least one deep learning model is selected from the group consisting of Visual Geometry Group (VGG), LeNet, AlexNet, U Net, and Residual Neural Network (ResNet).

31. The system of claim 28, wherein the at least one deep learning model is VGG-13.

32. The system of claim 28, further comprising a processing module configured to process said one or more digital images of the sperm sample by an unsupervised machine learning clustering algorithm.

33. The system of claim 28, wherein the unsupervised machine learning algorithm is selected from the group consisting of a k-means clustering algorithm, a gaussian mixture model, hierarchical clustering, spectral clustering, and k-medoids clustering.

34. The system of claim 28, further comprising means for staining the at least one sample of ZP-bound spermatozoa and the at least one sample of ZP-unbound spermatozoa prior to obtaining the one or more digital images using the means for obtaining the one or more digital images.

35. The system of claim 28, further comprising a cycle generative adversarial network (CycleGAN) module configured to perform CycleGAN to on said digital images of ZP-unbound and ZP-bound spermatozoa prior to implementing the at least one deep learning model.

36. The system of claim 28, wherein the-a threshold value to distinguish between normal and defective ZP-binding ability of spermatozoa is about 4.78%.

37. The system of claim 28, wherein the ZP-unbound spermatozoa were obtained from clinical samples with defective ZP-binding ability, wherein said clinical samples failed conventional insemination during-IVF in vitro fertilization (IVF).

38. The system of claim 28, wherein the ZP-bound spermatozoa were obtained by ZP-spermatozoa coincubation wherein the motile spermatozoa were incubated with one or more oocytes for 30 minutes to retrieve spermatozoa capable of binding to the ZP.

39. The system of claim 28, wherein said deep learning model VGG-13 predicts ZP-binding ability with at least about 90 % accuracy, specificity, sensitivity, or a combination thereof.

40. The system of claim 28, wherein the ZP-binding ability is predicted with specificity or sensitivity of at least about 90%.

41. The system of claim 28, wherein the ZP-binding ability is predicted with specificity or sensitivity of at least about 95%.

42. The system of claim 28, further comprising a prediction module configured to predict in vitro fertilization (IVF) success of spermatozoa based on their ZP-binding ability, wherein sperm samples with normal ZP-binding ability predict successful IVF, and those with defective ZP-binding ability predict unsuccessful IVF.

43. A system of predicting fertilization success following conventional insemination during-IVF in vitro fertilization (IVF), comprising:
a. means for obtaining a sperm sample from a subject undergoing IVF;
b. means for obtaining one or more digital images of said sperm sample through one or more imaging modalities; and
c. the deep learning-based system of claim 28 configured to obtain a binary prediction regarding ZP-binding ability of said sperm sample, wherein normal ZP-binding of said sperm sample predicts fertilization success, and defective ZP-binding of said sperm sample predicts fertilization failure.

44. The system of claim 28, wherein the at least one deep learning model is transfer learning.

45. The system of claim 28, wherein the at least one deep learning model is selected from the group consisting of Visual Geometry Group (VGG), LeNet, AlexNet, U Net, and Residual Neural Network (ResNet).

46. The system of claim 28, wherein the deep learning model is VGG-13.

47. The system of claim 28, further comprising a processing module configured to process said digital images of the sperm sample by an unsupervised machine learning clustering algorithm.

48. The system of claim 28, wherein the unsupervised machine learning algorithm is selected from the group consisting of a k-means clustering algorithm, a gaussian mixture model, hierarchical clustering, spectral clustering, and k-medoids clustering.

49. The system of claim 28, further comprising means for staining the sperm sample prior to obtaining the one or more digital images of the sperm sample using the means for obtaining the one or more digital images of the sperm sample.

50. The system of claim 28, wherein a threshold value to distinguish between normal and defective ZP-binding ability of the test sperm sample is about 4.78% ZP-binding.

51. The method of claim 28, wherein said VGG-13 model predicts ZP-binding ability with at least 90% accuracy, specificity, sensitivity, or a combination thereof.

52. The system of claim 28, wherein the ZP-binding ability is predicted with a specificity or sensitivity of at least about 90%.

53. The system of claim 28, wherein the ZP-binding ability is predicted with a specificity or sensitivity of at least about 95%.

54. A system for predicting the ZP-binding ability of spermatozoa, comprising:
a. means for obtaining at least one sample of ZP-bound spermatozoa and at least one sample of ZP-unbound spermatozoa;
b. means for obtaining one or more digital images of said ZP-bound and ZP-unbound spermatozoa through one or more imaging modalities; and
c. a hardware-based processor;
d. a memory configured to store instructions and configured to provide the instructions to the hardware-based processor; and
e. a set of modules configured to implement the instructions provided to the hardware-based processor, the set of modules including:
a deep learning module configured to implement at least one deep learning model, to process the one or more digital images according to at least one deep learning model configured to use sperm-the one or more digital images, and to generate a binary prediction regarding ZP-binding ability of spermatozoa, wherein the binary prediction is normal or defective ZP binding;
wherein said deep learning model is VGG-13 which predicts ZP-binding ability with at least about 90 % accuracy, specificity, sensitivity, or a combination thereof.

55. The system of claim 28, further comprising:
a classifier including, in sequence:
a first linear layer;
a second linear layer;
a rectified linear unit (ReLU) layer; and
a dropout layer.

56. claim 1, further comprising, prior to step b, the steps of:
selecting normospermic samples from the obtained samples of spermatozoa according to a predetermined criteria;
isolating motile and viable spermatozoa from seminal plasma;
incubating the isolated spermatozoa in a predetermined solution; and
staining the incubated spermatozoa.

57. The method of claim 1, further comprising, prior to step c:
receiving a training dataset; and
training the at least one deep learning model using the training dataset.
